(19) United States
(12) Patent Application Publication
(10) Pub. No.: US 2017/0014166 A1
(43) Pub. Date: Jan. 19, 2017

(54) AN IMPLANTABLE DEVICE

(71) Applicant: THE SYDNEY CHILDREN'S HOSPITALS NETWORK (RANDWICK AND WESTMEAD), Westmead, New South Wales (AU)

(72) Inventor: David Little, Westmead (AU)

(21) Appl. No.: 15/120,698

(22) PCT Filed: Feb. 23, 2015
(86) PCT No.: PCT/AU2015/050068

§ 371 Date: Aug. 22, 2016

(30) Foreign Application Priority Data
Feb. 21, 2014 (AU) 2014900570

(51) Int. Cl. A61B 17/72; A61L 27/12; A61L 27/58; A61L 27/18; A61B 17/88; A61B 17/70

(52) U.S. Cl. CPC A61B 17/72; A61B 17/8816; A61B 17/7098; A61L 27/58; A61L 27/18; A61L 27/12; A61B 2017/561

(57) ABSTRACT

An intramedullary bone device which has an internal lumen defined by a sidewall is described. The sidewall includes first and second perforated regions having respective first and second openings. The device also includes fluid flow directing features to direct the flow of a fluid from the internal lumen of the device through the openings in the sidewall of the device. The device may be part of a system with a fluid introducer member inserted through the internal lumen of the device. A method of securing and/or stabilising a bone including a fractured bone is also described.

### CROSS-REFERENCE TO RELATED APPLICATIONS

The present application claims priority from Australian Provisional Patent Application No 2014900570 filed on 21 Feb. 2014, the content of which is incorporated herein by reference.

### TECHNICAL FIELD

The disclosure relates to a device, system and method for providing strength to, or stabilising a bone and particularly a fractured bone during the healing process. Other apparatus and methods include delivery of agents to a bone to promote bone growth or repair.

### BACKGROUND

Many advances have been made in bone fixation methods in the last 50 years. However there is an ongoing need to reduce the morbidity and increase effectiveness of treatments by minimally invasive techniques. Metal rods can now be placed in the medullary canal for many fractures with standard devices in a minimally invasive way (often termed “closed” methods to differentiate them from “open” surgery). Even with plate technology, plates can be placed sub-muscularly and reduction effected and held by inserting the screws percutaneously through very small incisions. However, in children, these devices require adaptation such that they do not interfere with growth, and further, the implants routinely require a further operation and general anaesthetic to remove said standard devices. Therefore a minimally invasive method for fixing fractures which does not require further routine surgery and general anaesthetic for routine removal of implants would be advantageous.

In adults, standard orthopaedic practice does not necessarily involve the removal of implants but in certain fractures non-invasive treatment may be required for optimal outcomes.

Further, in delivering active bone agents to the skeleton to effect bone healing or spinal fusion, current clinical standards are open introduction of collagen based scaffolds containing the agent. Examples include Infuse® Bone graft (rhBMP-2 on a collagen sponge) and OP-1® or Ossigraft® (BMP-7 in granular collagen). Neither of these products can currently be delivered non-invasively by injection in clinical practice. One problem is maintaining the active element at the site. Therefore a delivery system and containment system that can be utilised non-invasively remain attractive areas for improvement in orthopaedic care.

### SUMMARY

In a first aspect, there is provided an intramedullary bone device extending from a first end to a second end and having a sidewall defining an internal lumen, said sidewall comprising a first perforated region at or adjacent to the first end and a second perforated region at or adjacent the second end, said first perforated region comprising first openings in the sidewall and the second perforated region comprising second openings in the sidewall, the first and second openings in fluid communication with the internal lumen, wherein the device further includes a first flow directing feature associated with at least one of the first openings and a second flow directing feature associated with at least one of the second openings and wherein the first flow directing feature directs the flow of a fluid from the internal lumen through the first openings in a first direction and the second flow directing feature directs the flow of a fluid from the internal lumen through the second openings in a second direction which is different to the first direction.

In a second aspect, there is provided a method of stabilising or securing a bone including;

preparing an entry point in the bone;

inserting a guidewire into the intramedullary canal of the bone;

reaming a length of the intramedullary canal over the guidewire;

advancing a device over the guidewire, the device extending from a first end to a second end and having a sidewall defining an internal lumen, said sidewall comprising a first perforated region adjacent the first end and a second perforated region adjacent the second end, the first perforated region comprising first openings in the sidewall and the second perforated region comprising second openings in the sidewall, the first and second openings in fluid communication with the internal lumen wherein the device further includes a first flow directing feature associated with the one or more first openings and a second flow directing feature associated with the one or more second openings;

withdrawing the guidewire when the device is desirably positioned within the bone;

introducing a fluid into the internal lumen of the device such that the first flow directing feature directs the flow of the fluid from the internal lumen through the first openings in a first direction and the second flow directing feature directs the flow of the fluid from the internal lumen through the second openings in a second direction which is different to the first direction.

In a third aspect, there is provided an intramedullary bone system comprising:

a device which extends from a first end to a second end and comprises a sidewall defining an internal lumen, said sidewall including a first perforated region adjacent said first end and a second perforated region adjacent the second end, said first perforated region comprising first openings in the sidewall and the second perforated region comprising second openings in the sidewall, the first and second openings in fluid communication with the internal lumen, wherein the device further includes a first flow directing feature associated with the one or more first openings and a second flow directing feature associated with the one or more second openings and wherein the first flow directing feature directs the flow of a fluid from the internal lumen through the first openings in a first direction and the second flow directing feature directs the flow of a fluid from the internal lumen through the second openings in a second direction which is different to the first direction;

a fluid introducer member positioned substantially within the internal lumen of the device, said fluid introducer member comprising a first end having an entry portal to receive the fluid, a second opposed end having at least one exit portal, and an internal channel fluidly connecting the entry and exit portals.

In a fourth aspect, there is provided a method of stabilising and/or securing a bone including;

preparing an entry point in the bone;

inserting a guidewire into the intramedullary canal of the bone;

reaming a length of the intramedullary canal of the bone over the guidewire;

advancing an intramedullary bone system over the guidewire, the intramedullary bone system comprising;

an intramedullary device extending from a first end to a second end and having a sidewall defining an internal lumen, said sidewall comprising a first perforated region adjacent the first end and a second perforated region adjacent the second end, the first perforated region comprising first openings in the sidewall and the second perforated region comprising second openings in the sidewall, the first and second openings in fluid communication with the internal lumen wherein the device further includes a first flow directing feature associated with the one or more first openings and a second flow directing feature associated with the one or more second openings; and

a fluid introducer member positioned substantially within the internal lumen of the intramedullary bone device, said fluid introducer member comprising a first end having an entry portal to receive the fluid, a second end having at least one exit portal, and an internal channel fluidly connecting the entry and exit portals and wherein the exit portal of the fluid introducer member is positioned within the second perforated region of the intramedullary bone device;

withdrawing the guidewire when the intramedullary bone system is desirably positioned within the bone;

introducing a fluid into the internal channel of the fluid introducer member such that said fluid flows from the exit portal of the fluid introducer member and is directed by at least one second flow directing feature through at least one second opening in a second direction;

withdrawing the fluid introducer member until the exit portal is positioned within the first perforated region of the intramedullary bone device such that the fluid flows from the exit portal of the fluid introducer member and is directed by at least one first flow directing feature through at least one first opening in a first direction, the first direction being different to the second direction.

In a fifth aspect, there is provided a bone stent comprising a series of struts defining apertures, the stent radially expandable from a compressed, undeployed configuration to an expanded configuration when deployed in a bone, wherein the stent extends from a proximal end to a distal end and further comprises a sealing member which substantially seals at least one of the proximal end or the distal end to substantially prevent leakage of any substance introduced into the stent, when in said deployed configuration.

In a sixth aspect, there is provided a system for stabilising a bone of a subject, said system including inserting a bone stent into a bone of a subject and applying a substance to the stent, wherein said bone stent comprises an elongate body having a main wall, the main wall comprising a series of struts defining apertures in the main wall, the stent radially expandable from a compressed, undeployed configuration to an expanded configuration when deployed in the bone, wherein the elongate body extends from a proximal end to a distal end and further comprising a sealing member at either or both ends, the sealing member substantially sealing at least one end of the stent to substantially prevent leakage of the substance from said end, when the stent is in said deployed configuration.

In a seventh aspect, there is provided an orthopaedic device comprising an introducer rod having an elongate body which extends from an open proximal end to a closed distal end, the introducer rod including a main wall which defines an internal lumen to receive a substance, said lumen extending from the open proximal end towards the distal end, the main wall also comprising a plurality of apertures therein.

In an eighth aspect, there is provided an orthopaedic delivery system, the delivery system comprising:

a carrier device having a main body configured for positioning in a subject, wherein the main body is expandable from a compressed, undeployed configuration to an expanded configuration when deployed in the subject; the main body having a wall comprising one or more apertures therein and wherein the main body defines an internal reservoir to receive a substance;

wherein the apertures of the main body have a size and/or a configuration such as to retain the substance in the main body for a period of time.

In a ninth aspect, there is provided an orthopaedic delivery system, the delivery system comprising:

a carrier device having a main body configured for positioning in a subject, said main body expandable from a compressed, undeployed configuration to an expanded configuration when deployed in the subject; the main body having a wall comprising one or more apertures therein and defining an internal reservoir;

a high viscosity liquid carrier material (HVLCM) in said internal reservoir, said HVLCM having a pre-determined viscosity;

wherein the main body is configured such that at least a portion of the apertures are sized and/or configured such that the HVLCM is substantially housed within the carrier device for period of time.

In a tenth aspect, there is provided a method of stabilising a bone of a subject including:

reaming an intramedullary channel through a bone;

introducing an introducer member carrying a stent into the channel, the stent comprising an elongate body having a main wall defining a lumen therethrough, the main wall comprising a series of struts defining apertures in the main wall, the stent radially expandable from a compressed configuration to an expanded configuration, wherein the elongate body extends from a proximal end to a distal end and further comprising a sealing member which substantially seals at least one of the proximal or the distal end, said stent introduced into the bone in its compressed configuration;

moving the introducer in a proximal direction to deploy the stent from the introducer;

causing or allowing the stent to move from its compressed configuration to its expanded configuration within the bone cavity;

introducing a stabilising substance into the lumen of the stent.

In an eleventh aspect, there is provided a method of introducing a carrier device and carrier substance to a subject including:

loading an introducer with a carrier device, said carrier device comprising a main body which is expandable from a compressed configuration to an expanded configuration, the main body comprises a main wall having one or more apertures of a pre-determined size and/or configuration defined therein and defining an internal reservoir, wherein said carrier device is loaded in the introducer in its compressed configuration;

percutaneously advancing the introducer to a desired site in a subject;

withdrawing the introducer to deploy the carrier device and cause or allow the carrier device to move from its compressed configuration to its expanded configuration;

removing the introducer and inserting a catheter percutaneously to said site, the catheter having an internal lumen to deliver a liquid carrier substance to the carrier device;

arranging the catheter relative to the carrier device such that the lumen of the catheter is in fluid communication with the reservoir of the carrier device;

delivering the liquid carrier substance to the reservoir;

wherein the liquid carrier substance has a pre-determined viscosity and the apertures of the carrier device are sized and/or configured such that the liquid carrier substance is substantially housed within the carrier device for at least a period of time.

### EXEMPLARY EMBODIMENTS OF THE DISCLOSURE

### Intramedullary Bone Device

The disclosures of the first and second aspects, relate to an intramedullary bone device which extends from a first end to a second end and has a sidewall defining an internal lumen. A method of implanting this device is also disclosed. A system comprising the intramedullary bone device and further including an introducer member is also disclosed in the third aspect and a method of introducing the system is disclosed in the fourth aspect.

The sidewall of the intramedullary device comprises a first perforated region adjacent the first end and a second perforated region adjacent the second end.

The first perforated region comprises one or more first openings in the sidewall and the second perforated region comprises one or more second openings in the sidewall. The first and/or second openings may be in fluid communication with the internal lumen.

The device further includes a first flow directing feature associated with the one or more first openings and a second flow directing feature associated with the one or more second openings.

The first flow directing feature directs the flow of a fluid from the internal lumen through the first openings in a first direction and the second flow directing feature directs the flow of a fluid from the internal lumen through the second openings in a second direction which is different to the first direction.

The intramedullary bone device is typically substantially tubular and extends along a first axis. The first and second perforated regions may be separated by an intermediate region, said intermediate region typically devoid of openings in the sidewall.

At least some, preferably all of the first openings may be spaced around the sidewall in an ordered and/or a random fashion. Typically, the first openings are offset relative to each other. In another embodiment of a tubular intramedullary bone device, at least some of the first openings are linearly and/or circumferentially aligned relative to each other around the sidewall. In one embodiment, at least some of the first openings are helically arranged in the sidewall of the intramedullary bone device.

At least some, preferably all of the second openings may be spaced around the sidewall in an ordered and/or a random fashion. Typically, the second openings are offset relative to each other. In another embodiment of a tubular intramedullary bone device, at least some of the second openings are linearly and/or circumferentially aligned relative to each other around the sidewall. In one embodiment, at least some of the second openings are helically arranged in the sidewall of the intramedullary bone device.

The first perforated region may comprise between 10 to 50% of the total length of the intramedullary bone device. Typically, the first perforated region extends between 20% and 40% of the total length of the intramedullary bone device. In one embodiment, the first perforated region comprises approximately 30% of the total length of the intramedullary bone device. The first perforated region may not extend as far as the first end such that a primary region of the intramedullary bone device adjacent the first end is devoid of openings.

The second perforated region may comprise between 10 to 50% of the total length of the intramedullary bone device. Typically, the first perforated region extends between 20% and 40% of the total length of the intramedullary bone device. In one embodiment, the second perforated region comprises approximately 30% of the total length of the intramedullary bone device. The second perforated region may not extend as far as the second end such that there is a terminal region of the intramedullary bone device between the second end and the second perforated region which is devoid of openings.

The intermediate region may comprise between 10 to 60% of the total length of the intramedullary bone device. Typically, the intermediate region extends between 20% and 40% of the total length of the device. In one embodiment, the intermediate region comprises approximately 30% of the total length of the intramedullary bone device. The intermediate region typically spans a length of the intramedullary bone device between the first and the second perforated regions.

A first flow directing feature may comprise part of the sidewall of the intramedullary bone device near an associated first opening. Alternatively, a first flow directing feature may comprise a separate member connected to part of the sidewall.

Similarly, a second flow directing feature may comprise part of the sidewall of the intramedullary bone device near an associated second opening. Alternatively, a second flow directing feature may comprise a separate member connected to part of the sidewall.

In one embodiment, part of the sidewall adjacent to a first and/or second opening extends into the internal lumen to form a first and/or second flow directing feature. In one embodiment, this may be achieved by punching a flap in a sidewall and urging the flap into the internal lumen of the intramedullary device. The flap may form or be further molded to form a first and/or second flow directing feature.

An exemplary first fluid guide member associated with a first opening may extend inwardly and at an angle relative to the sidewall of the intramedullary bone device. The angle may vary and the particular angle at which a first fluid guide member extends relative to the sidewall determines the trajectory of the fluid as it exits an associated first opening.

A first fluid guide member may extend internally a distance of between 10% and 60% of the overall diameter of the intramedullary bone device. Typically, a first fluid guide member extends a distance between 20% and 40% of the diameter of the device. In one embodiment, a first fluid guide member extends a distance of around 30% of the diameter of the device.

A first fluid guide member may be partially circular in shape. Alternatively, a guide member may be oval, square, rectangular or any other shape as determined by the cut in the sidewall.

A first fluid guide member typically depends from a connection end connected to the sidewall. The connection end may comprise a superior edge of the first opening, that is, part of the sidewall defining the opening which is closer to the first end of the intramedullary bone device. In this embodiment, the first fluid guide member depends towards the second end of the intramedullary bone device. The first fluid guide member may depend at an angle as noted above relative to the sidewall and towards the second end of the intramedullary bone device such that it spacedly extends down over an associated first opening to define a flow channel from the internal lumen to the associated first opening.

A first fluid guide member may be substantially planar along its length from the connection end to a second end free end. Alternatively the first fluid guide member has a width and is substantially curved across its width. Further, a first guide member may be substantially curved along its length.

The connection end of a first fluid guide member may comprise a relatively small percentage of the overall circumference of the sidewall of the intramedullary bone device defining an associated first opening, such as between 5% to 10% of the overall circumference. Alternatively, the connection end may extend between approximately 10% to 20%; or 20% to 30%; 40% to 50%; 50% to 60%, or 70% to 80% of the overall circumference of the sidewall defining the associated opening.

A plurality of first fluid guide members may be arranged internally in the first perforated region of the device. Each fluid guide member is typically associated with a first opening.

The first fluid guide members may together define a narrower passage within the internal lumen of the first perforated region. In one embodiment, the free second ends of a plurality of first fluid guide members may be shaped such that they define the passage.

This embodiment may be useful when inserting an introducer rod or other device to deliver the fluid. In this embodiment, the narrower passage is relatively central and thus centralizes the introducer rod within the intramedullary bone device. An advantage of having a centralized introducer rod is that the fluid is substantially evenly distributed from the introducer rod to the intramedullary bone device and thereafter to the bone.

The first fluid guide members may be substantially identical in shape and/or orientation relative to each other to provide a uniform flow pattern of fluid therethrough. Alternatively, the first fluid guide members may comprise different shapes relative to each other and/or may extend at different angles relative to the sidewall.

An exemplary second fluid guide member associated with a second opening may extend inwardly and at an angle relative to the sidewall of the intramedullary bone device. The angle may vary and as with the first fluid guide members, the particular angle at which a second fluid guide member extends relative to the sidewall determines the trajectory of the fluid as it exits an associated second opening.

A second fluid guide member may extend internally a distance of between 10% and 60% of the overall diameter of the intramedullary bone device. Typically, a second fluid guide member extends a distance between 20% and 40% of the diameter of the device. In one embodiment, a second fluid guide member extends a distance of around 30% of the diameter of the device.

A second fluid guide member may be partially circular in shape. Alternatively, a guide member may be oval, square, rectangular or any other shape as determined by the cut in the sidewall. The shape of at least some of the second fluid guide members may be the same as at least some of the first fluid guide members. Alternatively the second fluid guide members may differ in shape relative to the first fluid guide members.

A second fluid guide member may extend from a connection end connected to the sidewall. The connection end may comprise an inferior edge of the second opening, that is, part of the sidewall defining the opening which is closer to the second end of the intramedullary bone device. In this embodiment, the second fluid guide member extends towards the first end of the intramedullary bone device. The second fluid guide member may extend at an angle sidewall and towards the first end of the intramedullary bone device such that it spacedly extends up over an associated second opening to define a flow channel from the internal lumen to the associated second opening.

A second fluid guide member may be substantially planar along its length from the connection end to a second end free end. Alternatively the second fluid guide member has a width and is substantially curved across its width. Further, a second guide member may be substantially curved along its length.

The connection end of a second fluid guide member may comprise a relatively small percentage of the overall circumference of the sidewall of the intramedullary bone device defining an associated second opening, such as between 5% to 10% of the overall circumference. Alternatively, the connection end may extend between approximately 10% to 20%; or 20% to 30%; 40% to 50%; 50% to 60%, or 70% to 80% of the overall circumference of the sidewall defining the associated opening.

A plurality of second fluid guide members may be arranged internally in the second perforated region of the device. Each fluid guide member is typically associated with a second opening.

The second fluid guide members may together define a narrower passage within the internal lumen of the second perforated region. In one embodiment, the free second ends of a plurality of second fluid guide members may be shaped such that they define the passage.

The narrower passage in the second perforated region may also be useful when inserting an introducer rod or other device to deliver the fluid such as to centralize the introducer rod within the second perforated region of the intramedullary bone device.

The second fluid guide members may be substantially identical in shape and/or orientation relative to each other to provide a uniform flow pattern of fluid therethrough. Alternatively, the second fluid guide members may comprise different shapes relative to each other and/or may extend at different angles relative to the sidewall.

The intramedullary bone device may be substantially circular in cross section. Alternatively, it may be oval, elliptical, rectangular, square or any other suitable cross section for positioning in a bone of a subject.

Typically, the intramedullary bone device is substantially straight. This is particularly the case for use in long bones. However, it is envisaged that the device may also be another shape depending upon the bone in which it is to be implanted. For example, the device may be curved in shape, have one or more flared ends or be tailored for any particular shape corresponding to a particular anatomy of a bone. In one embodiment, the primary portion of the intramedullary bone device, adjacent the first end, is angled relative to the remainder of the intramedullary bone. This embodiment may be used for trochanteric entry of the femur.

In a further embodiment, the device is made from a substantially flexibly resilient material such that it may conform to the shape of a bone in which it is deployed.

Typically the intramedullary bone device of this disclosure is made from a polymeric material. A polymeric intramedullary bone device may be prepared by conventional methods such as extrusion, injection molding, and solvent casting to form the desired shape and configuration and size of the first and second openings.

The polymer of the intramedullary bone device may include materials such as conventional biocompatible, resorbable or nonresorbable polymers. In a preferred embodiment, the intramedullary bone device is made from a biocompatible resorbable polymer.

Examples of biocompatible, resorbable polymers include poly(α-hydroxy esters) such as poly(lactide), poly(glycolide), poly(caprolactone), poly(p-dioxanone), poly(trimethylene carbonate), poly(oxaesters), poly(oxaamides), and their co-polymers and blends. “Poly(glycolide)” is understood to include poly(glycolic acid). “Poly(lactide)” is understood to include polymers of L-lactide, D-lactide, meso-lactide, blends thereof, and lactic acid polymers.

Other examples of suitable resorbable polymers include tyrosine derived poly amino acid such as poly(DTH carbonates), poly(arylates), and poly(imino-carbonates)], phosphorous containing polymers such as poly(phosphoesters) and poly(phosphazenes), poly(ethylene glycol) [PEG] based block co-polymers PEG-PLA, PEG-poly(propylene glycol), PEG-poly(butylene terephthalate)], poly(α-malic acid), poly(ester amide), and polyalkanoates such as poly(hydroxybutyrate (HB) and poly(hydroxyvalerate) (HV) co-polymers. Co-polymers of poly(lactide), and poly(glycolide) (PLGA) may also be desirable, wherein in one embodiment, the proportion of each polymer may vary to modulate strength and resorption time in the body. Furthermore, trimethyl carbonate may be blended with any of the above polymers. For example the intramedullary bone device may include up to 30% w/w of trimethyl carbonate with any of the above polymers.

Examples of non-resorbable polymers include polyolefins, polyamides, polyesters, fluoropolymers, and acrylics.

The intramedullary bone device may comprise blends of polymeric materials or blends of polymeric materials and plasticizers. The polymeric materials may also be blended with additives for osteoconduction and/or with agents to change the pH profile as the intramedullary bone device resorbs.

In one embodiment the intramedullary bone device may further comprise bioglass, hydroxyapatite, calcium carbonate. Any suitable biocompatible material to increase the pH of the resorbing polymer may be added to the intramedullary bone device

It is envisaged that the intramedullary bone device may alternatively be made from a metal or a metal alloy. The metal or metal alloy may be resorbable. An example includes a magnesium alloy such as a magnesium zinc calcium alloy.

The diameter of the intramedullary bone device will typically depend upon the reamed diameter of the intramedullary bone cavity in which the device is deployed.

The intramedullary bone device may also include therapeutic agents, radiopaque agents or both.

The therapeutic agents may be incorporated into the device by coating the intramedullary bone device or impregnating the material of the intramedullary bone device during manufacture.

A radiopaque agent may be impregnated into the material of the intramedullary bone device or, alternatively, coated onto an exterior of the device.

The radiopaque agent is typically selected from one or more biocompatible agents. Examples of suitable agents include bismuth oxides, barium sulfate and/or iodine compounds. The radiopaque additives may alternatively include metal powders such as tantalum, tungsten or gold, or metal alloys having gold, platinum, iridium, palladium, rhodium, or a combination thereof.

The fluid described herein may be resorbable or alternatively non-resorbable. In one embodiment, the fluid comprises a bone cement. The bone cement may be a resorbable bone cement. An example of a suitable resorbable bone cement includes calcium. One example includes calcium phosphate. Further examples include dicalcium phosphate, tricalcium (α and β) phosphate, tetracalcium phosphate hydroxyapatite or a mixture of two or more thereof. Still further, the bone cement may include one or more of the following: heptacalcium phosphate, octocalcium phosphate, calcium pyrophosphate, oxyapatite, calcium metaphosphate, dahlite, carbonatoapatite, monocalcium phosphate anhydrous, amorphous calcium phosphate, calcium deficient hydroxyapatite, fluorapatite; a calcium silicate ceramic that includes: calcium orthosilicate, wollastonite, dicalcium silicate, diopside, and bioglass (any composition); or a calcium salt such as calcium sulfate (α-calcium sulfate hemihydrates, β-calcium sulfate hemihydrates, calcium sulfate dehydrate and mixtures thereof), plaster of paris; or a mixture thereof. A portion of the calcium content in the ceramic may be replaced by another divalent cation such as magnesium or strontium.

In one embodiment, the pH profile of the resorbing cement has a pH which is basic. For example the resorbing cement may have a pH greater than 7.2.

The bone cement may, alternatively, be non-resorbable. Examples of non-resorbable bone cements include methylmethacrylate and methyl acrylate or a mixture thereof.

The bone cement may be introduced from the first end of the resorbable bone cement device in a relatively flowable state such that it migrates through the internal lumen towards the second end of the intramedullary bone device.

The cement may be selected for various properties including an optimal mixing, delivery and setting time. Mixing Time may range from 30 seconds to 2 minutes and preferably 30 seconds to one minute. Delivery time, that is, how long the cement is in liquid form for delivery to the desired site may range from 2 minutes to 5 minutes. Preferably the delivery time is 3 to 4 minutes. Setting time wherein the cement substantially hardens within the body may be from 2 to 20 minutes. Preferably the setting time may be 4 to 10 minutes. By the term “harden” it should be appreciated that the cement may continue to harden in the body for further periods of time beyond the setting time. The term “harden” is used to describe the substantial hardening beyond the flowable form of the liquid state such that the cement can hold its shape at the site of delivery.

In use, the intramedullary bone device may stabilise a fracture in a bone of a subject. Examples of bone fractures include long bone fractures such as to the femur, tibia, fibula, humerus, radius and ulna; and other fractures such as to the metacarpal bones of the hand and metatarsal bones of the feet, the clavicle. Other uses are envisaged such as to stabilise metastatic bone or other bone having lesions therein.

An advantage of providing both a bioresorbable intramedullary bone device to receive a bioabsorbable bone cement is that further surgery to remove the device and cement is not required. This may be particularly useful in stabilising or correcting a fracture but in other applications of stabilisation such as in the metastatic bone, the device and cement may be left in the bone.

A method of securing a fracture includes preparing an entry point in the bone and reaming a desired length of the intramedullary canal of the fractured bone. A guidewire may be inserted and the fracture reduced.

In one embodiment, prior to insertion, an introducer rod is inserted into the first end of the intramedullary bone device and passed through the internal lumen and towards the second end of the intramedullary bone device.

The introducer rod typically includes an entry portal at one end to receive a fluid such as bone cement and at least one exit portal adjacent a second end. The introducer rod includes an inner channel connecting the entry portal and the at least one exit portal. In one embodiment, the at least one exit portal comprises a plurality of holes in a sidewall of the introducer rod adjacent the second end of the introducer rod. In another embodiment, the at least one exit portal comprises an aperture in the second end of the introducer rod. Still further, the at least one exit portal may comprise both an aperture in the end of the introducer rod and one or more holes in the sidewall adjacent said second end of the introducer rod.

The introducer rod may be inserted into the intramedullary bone device until the at least one exit portal is positioned in the second perforated region.

The assembly of the intramedullary bone device and the introducer rod may be inserted into the bone by inserting the second end of the intramedullary bone device and the second end of the introducer rod over the guidewire and into the intramedullary canal of the bone until said second end is positioned beyond the fracture site. Depending upon the bone and the point of entry, the second end of the intramedullary bone device may be positioned either proximal or distal to the fracture site. Typically, the positioning should be such that the second perforated region and the first perforated region of the intramedullary bone device are positioned on either side of a fracture site.

The intermediate region of the intramedullary bone device may be positioned adjacent to the fracture site. Because the intermediate region is devoid of openings, substantially no injected cement enters the fracture site when injected into the intramedullary bone device.

With the device appropriately positioned relative to the fracture site, the guidewire may be removed and a cement source connected to the entry portal of the introducer rod which is in situ within the internal lumen of the intramedullary bone device.

Typically, the cement source includes a cement gun or like device which is connectable to the entry portal. In this embodiment, the entry portal of the introducer rod may include a sealing member to seal a nozzle of the cement gun or like device therein. An example may include a Luer lock at the entry portal.

The cement may flow through the internal channel of the introducer rod before exiting the at least one exit portal. Upon exiting the at least one exit portal, the cement enters the internal lumen of the intramedullary bone device and is guided by the second guiding members through their associated second openings. The orientation of the second guiding members is such that the bone cement is guided towards the second end of the device, and importantly, guided away from the fracture site.

In one embodiment, an intra-osseous needle or similar device may be inserted through the internal lumen of the intramedullary bone device. The needle may be connected to a negative pressure source to provide a suction pressure in the internal lumen. When inserted further towards the second end of the intramedullary device relative to the second end of the introducer rod, such suction pressure assists the flow of cement from the exit portal of the introducer rod and through the first and/or second openings of the intramedullary bone device.

The introducer rod may be gradually retracted towards the first end of the intramedullary bone device.

Typically, the introducer rod is made from or includes a radiopaque material and as such its positioning may be visualised by a user.

Once the introducer rod is retracted to the first perforated region, as the cement exits the exit portal of the introducer rod and enters into the internal lumen of the intramedullary bone device, the first guiding members may guide the flow of cement through associated first openings and again, due to the orientation of the first fluid guide members, the cement typically flows through the first openings and away from the fracture site.

As mentioned above, the arrangement of internally extending first and second fluid guide members in the intramedullary bone device provides a relatively central passage through which the introducer rod may pass. The resultant device/rod assembly is relatively stiffer than the individual components and therefore this embodiment protects the intramedullary bone device from damage or breakage as it is moved into the intramedullary canal or further adjusted within the intramedullary canal.

The introducer rod may be removed fully from the intramedullary bone device and the fracture held until the cement has substantially set. The setting time wherein the cement substantially hardens within the body may vary but as noted above can be from 2 to 20 minutes. Preferably the setting time may be 4 to 10 minutes.

The first and second fluid guide members of the intramedullary bone device may provide an increased surface area for bonding with a bone cement, thus strengthening the overall device/cement scaffold in the bone of a subject to stabilise said bone.

### Introducer Rod

The introducer rod disclosed may be part of a reinforced cement system which is left in situ within the bone such as described above for the intramedullary bone device. Alternatively, the introducer rod may be used to deliver a substance to the herein disclosed stent. One or more of the features of the introducer rod described herein may relate to either application.

The elongate rod may comprise a tubular structure defining a lumen which extends from the proximal end to the distal end. The introducer rod may be substantially circular in cross section. Alternatively, the introducer rod may be oval, elliptical, rectangular, square or any other suitable cross section for positioning in a bone of a subject.

The main wall of the introducer rod typically comprises a series of apertures from an inner surface and extending to an outer wall such that the substance introduced into the rod may move from the lumen and through the apertures.

In the context of inserting the introducer rod into a fractured bone, in one embodiment, the main wall may include a length which is devoid of any apertures. It is envisaged that when in position within a bone, the region of the main wall which has no apertures will be positioned adjacent the fracture site. With no apertures in this part of the rod, the substance being introduced will not leak through the fractured bone and into surrounding area of tissue.

The apertures in the elongate body may be arranged in a number of configurations along the elongate body. For example, the apertures may be randomly distributed. More preferably, the apertures may be configured in an order or a pattern in the main wall. In one embodiment, the apertures may be distributed in a helical pattern along a length of the elongate body. This embodiment may allow for an optimal distribution of the bone agent. The apertures may be uniformly sized. Alternatively some of the apertures may be of different size to other apertures. The shape of the apertures may also be uniform or may vary.

The introducer rod may be rotatably moveable. In this embodiment, the proximal end of the elongate body may be connected to a motor to drive rotation around a longitudinal axis of the introducer rod. In an embodiment having a helical arrangement of the apertures, rotation of the introducer rod may create an optimal “spray” of the substance to the surrounding area.

The introducer rod may also be coupled to a substance source. The lumen of the introducer rod is typically in fluid communication with the source. In the embodiment where the substance comprises a bone cement, the substance source may be a source of suitable cement in a substantially liquid state. The device may further comprise an actuator member to cause the flow of the cement from the cement source and into the lumen of the introducer rod. The motor may also be actuated by the actuator member such that the introducer rod rotates as the cement is introduced. Such an embodiment may prevent premature setting of the cement in the lumen of the rod before the cement has traveled through the apertures and engages the surrounding bone surface, adjacent the apertures.

Once the cement has spread to the desired regions within the rod and through the apertures to cement with the surrounding bone, and once the cement has substantially set, the elongate rod is typically left in the bone. One or both of the introducer rod and the cement are resorbable. Alternatively, one or both of the introducer rod and cement may be non-resorbable.

In one embodiment, the introducer rod is relatively rigid. However, it is also envisaged that the introducer rod may be suitably flexible such that in a deployed configuration it substantially conforms to the surface or into a cavity of a bone or other part of the body in which it is deployed.

The introducer rod may be made from a metal, a metal alloy or a polymeric material. The diameter of the introducer rod and the thickness of the wall for both non-polymeric and polymeric introducer rods will typically depend upon the diameter of the intramedullary cavity of a bone in which it is deployed.

The introducer rod may be made from a number of different biocompatible, bioabsorbable or nonabsorbable polymers.

Examples of non-absorbable polymers include polyolefins, polyamides, polyesters, fluoropolymers, and acrylics. Examples of biocompatible, resorbable polymers include poly(α-hydroxy esters) such as poly(lactide), poly(glycolide), poly(caprolactone), poly(p-dioxanone), poly(trimethylene carbonate), poly(oxaesters), poly(oxaamides), and their co-polymers and blends. “Poly(glycolide)” is understood to include poly(glycolic acid). “Poly(lactide)” is understood to include polymers of L-lactide, D-lactide, meso-lactide, blends thereof, and lactic acid polymers.

Other examples of suitable resorbable polymers include tyrosine derived poly amino acid such as poly(DTH carbonates), poly(arylates), and poly(imino-carbonates)], phosphorous containing polymers such as poly(phosphoesters) and poly(phosphazenes), poly(ethylene glycol) [PEG] based block co-polymers PEG-PLA, PEG-poly(propylene glycol), PEG-poly(butylene terephthalate)], poly(α-malic acid), poly(ester amide), and polyalkanoates such as poly(hydroxybutyrate (HB) and poly(hydroxyvalerate) (HV) co-polymers.

The polymeric material may also comprise blends of polymeric materials or blends of polymeric materials and plasticizers.

The introducer rod may also include therapeutic agents, radiopaque agents or both. The therapeutic agents may be incorporated into introducer rod by coating the introducer rod or impregnating the material of the introducer rod during manufacture.

Similarly, the radiopaque agent may be impregnated into the material of the rod or, alternatively, coated onto an exterior of the rod. The radiopaque agent may be selected from one or more biocompatible agents. Examples of suitable agents include bismuth oxides, barium sulfate and/or iodine compounds. The radiopaque additives may alternatively include metal powders such as tantalum, tungsten or gold, or metal alloys having gold, platinum, iridium, palladium, rhodium, or a combination thereof.

The bone cement of the present disclosure which is introduced into the introducer rod may comprise a resorbable cement or alternatively a non-resorbable cement. Examples of a suitable resorbable bone cement include calcium phosphate. Further examples include calcium sulphate, dicalcium phosphate, tricalcium phosphate, tetracalcium phosphate hydroxyapatite or a mixture of two or more thereof.

Examples of non-resorbable bone cements include methylmethacrylate and methyl acrylate or a mixture thereof.

The use of the introducer rod as herein disclosed may be of particular use in a bone which has a narrow diameter and in which it is not possible to introduce an expandable stet or other device.

### Bone Stent

The bone stent may comprise a tubular structure defining a lumen which extends from the proximal end to the distal end. The bone stent may be substantially circular in cross section. Alternatively, the bone stent may be oval, elliptical, rectangular, square or any other suitable cross section for positioning in a bone of a subject.

The elongate body of the stent may comprise a substantially tubular member with a substantially circular cross section when in its compressed, undeployed configuration. When deployed and in its expanded configuration, the elongate body may take on a different shape to that of the compressed shape.

Typically, in its deployed configuration, the elongate body is substantially straight, particularly for use in long bones. However, it is envisaged that the stent elongate body may also be serpentine in shape. Alternatively, the elongate body may be curved in shape. In one embodiment, it is envisaged that a region adjacent one or both of the proximal or distal ends defines a tubular structure having a greater diameter than the remainder of the elongate body. In this embodiment, the elongate body may substantially flare outwardly at one or both ends. A trumpet shaped device may be particularly useful in surgeries of bones such as the clavicle. Other configurations of the final deployed configuration are, however, envisaged such as an elongate body with a wider diameter in a middle section and tapering towards the proximal and distal ends. The elongate body may be tailored for any particular shape corresponding to a particular anatomy of a bone.

The expanded shape may be pre-determined and the stent primed to take on such a pre-determined shape when deployed in the bone. A stent made from a shape memory material may be suitable for such an embodiment and in instances where the shape of the bone is known. By tailoring the deployed stent to the shape of the bone or a cavity of a bone, a better fit within the bone is achieved.

Examples of suitable shape memory materials include several metal alloys. One example is a copper-aluminium-nickel alloy. Furthermore, a nickel-titanium (NiTi) alloy such as Nitinol may be used to form the stent. Other examples include alloys of zinc, copper, gold and iron.

A particular shape of the stent in a deployed configuration may also be achieved by the arrangement or configuration of struts in the main wall of the elongate body. Together, the struts of the stent are typically arranged to define a series of cells which provide tensile strength to the stent while also defining apertures in the main wall.

In one embodiment, a cell comprises a diamond shaped structure. In another embodiment, the cells may define a triangular, square or rectangular, structure.

In one embodiment each cell may comprise multiple struts, including two, three, four, five, six, seven, eight struts or more.

The cells of the stent may all be the same in size and/or configuration or may differ in size and/or configuration. For example, all the cells of the stent may have the same number of sides/struts. Alternatively, a proportion of the cells of the stent may differ in the number of sides/struts relative to the other cells of the stent.

The struts of cells may be relatively straight. Alternatively, the struts may be curved or sinusoidal.

Typically, the cells are circumferentially arranged around the stent main wall in a series. Desirably, at least one cell in one circumferentially arranged series is connected to or integral with an adjacent cell in another circumferential series. At least one cell may have at least one common side with another cell in said circumferential series or adjacent circumferential series.

Typically, the entire length of the stent comprises a plurality of circumferentially arranged series of cells.

To vary the features of the stent such as strength and/or the shape, the cell arrangement may be varied such as by varying the size of the cells i.e. the size of the aperture formed by the sides of the cells. For example, at least a portion of the length of the elongate body may comprise a series of relatively closely spaced cells defining relatively small apertures. A further portion of the main wall of the elongate body may comprise a more open cell arrangement with the struts defining relatively larger sized apertures.

In a further embodiment, the stent elongate body may be suitably flexible such that in a deployed configuration it substantially conforms to the surface of a bone or other part of the body in which it is deployed.

As also noted, the stent may alternatively be made from a metal, a metal alloy or a polymeric material. A polymeric stent may be prepared by conventional methods such as extrusion, injection molding, and solvent casting to form the desired shape and configuration of cells. A metal or metal alloy stent may be made from a metal tube (eg Nitinol, or stainless steel) which has been etched or more preferably laser cut to the desired configuration of cells.

The diameter of the stent and the thickness of the wall for both non-polymeric and polymeric stents may depend upon the desired final diameter of the stent, which is in turn dependent on the diameter of the intramedullary bone cavity in which the stent will be deployed.

The polymer of the bone stent may include polymeric materials such as conventional biocompatible, bioabsorbable or nonabsorbable polymers. The selection of the polymeric material used to prepare the bone stent may be selected according to multiple factors such as absorption times and physical properties of the materials, and the geometry of the bone stent.

Examples of non-absorbable polymers include polyolefins, polyamides, polyesters, fluoropolymers, and acrylics.

Examples of biocompatible, resorbable polymers include poly(α-hydroxy esters) such as poly(lactide), poly(glycolide), poly(caprolactone), poly(p-dioxanone), poly(trimethylene carbonate), poly(oxaesters), poly(oxaamides), and their co-polymers and blends. “Poly(glycolide)” is understood to include poly(glycolic acid). “Poly(lactide)” is understood to include polymers of L-lactide, D-lactide, meso-lactide, blends thereof, and lactic acid polymers.

Other examples of suitable resorbable polymers include tyrosine derived poly amino acid such as poly(DTH carbonates), poly(arylates), and poly(imino-carbonates)], phosphorous containing polymers such as poly(phosphoesters) and poly(phosphazenes), poly(ethylene glycol) [PEG] based block co-polymers PEG-PLA, PEG-poly(propylene glycol), PEG-poly(butylene terephthalate)], poly(α-malic acid), poly(ester amide), and polyalkanoates such as poly(hydroxybutyrate (HB) and poly(hydroxyvalerate) (HV) co-polymers. Co-polymers of poly(lactide), and poly(glycolide) (PLGA) may also be desirable, wherein in one embodiment, the proportion of each polymer may vary to modulate strength and resorption time in the body.

The polymeric material may also comprise blends of polymeric materials, blends of polymeric materials and plasticizers.

In both non-polymeric and polymeric embodiments, the stent elongate body may also include therapeutic agents, radiopaque agents or both.

The therapeutic agents may be incorporated into the stent by coating the stent or impregnating the material of the stent during manufacture.

The stent may, as mentioned include a radiopaque agent. As with the therapeutic agent, the radiopaque agent may be impregnated into the material of the stent or, alternatively, coated onto an exterior of the stent.

The radiopaque agent is typically selected from one or more biocompatible agents. Examples of suitable agents include bismuth oxides, barium sulfate and/or iodine compounds. The radiopaque additives may alternatively include metal powders such as tantalum, tungsten or gold, or metal alloys having gold, platinum, iridium, palladium, rhodium, or a combination thereof.

The bone stent may include more than one elongate body. In one embodiment, a first elongate body comprises an inner stent and is at least partially surrounded by a second elongate body forming an outer stent. The inner and outer stents may be connected to one another by one or more radial connector struts. The distance between the inner and outer stents may vary depending upon the length of the radial struts.

Rather than separate elongate bodies, alternatively the main wall of the elongate body may have a thickness with an inner surface defining a lumen therethrough and an outer surface configured for engagement with the sealing member and/or a surface of bone in which the stent is deployed. The inner surface and the outer surface of the stent may be interconnected by radially extending struts within the main wall.

Whether as a single stent or two or more stents radially connected, the radial struts may be configured to facilitate the compression of the stent into the compressed configuration for delivery into a bone and the subsequent radial expansion upon deployment in the bone.

The elongate body of the stent may also be longitudinally reinforced by one or more longitudinally extending reinforcement members.

The stent of the present disclosure is configured to receive substance. An example of a substance for use with the bone stent includes a bone cement. The substance may be resorbable or alternatively non-resorbable. In one embodiment, the substance comprises a resorbable bone cement. An example of a suitable resorbable bone cement includes calcium phosphate. Further examples include dicalcium phosphate, tricalcium phosphate, tetracalcium phosphate hydroxyapatite or a mixture of two or more thereof.

Examples of non-resorbable bone cements include methylmethacrylate and methyl acrylate or a mixture thereof.

The bone agent may be introduced from the proximal end of the elongate body or at any other region along the elongate body. Typically, the bone agent is in a relatively flowable state when introduced into the stent such that it substantially fills both the lumen of the stent and also migrates through the apertures in the main wall.

In the embodiment wherein the bone agent is a bone cement, the cement and the stent provide a reinforced structure which provides both tensile strength from the reinforcement struts of the stent and compressive strength from the cement, to a bone in which the stent and cement are deployed.

The cement is selected for various properties including an optimal mixing, delivery and setting time. Mixing Time may range from 30 seconds to 2 minutes and preferably 30 seconds to one minute. Delivery time, that is, how long the cement is in liquid form for delivery to the desired site may range from 2 minutes to 5 minutes. Preferably the delivery time is 3 to 4 minutes. Setting time wherein the cement substantially hardens within the body may be from 2 to 20 minutes. Preferably the setting time may be 4 to 10 minutes. By the term “harden” it should be appreciated that the cement may continue to harden in the body for further periods of time beyond the setting time. The term “harden” is used to describe the substantial hardening beyond the flowable form of the liquid state such that the cement can hold its shape at the site of delivery.

The sealing member typically comprises a sheath which is configured to surround at least part of the stent elongate body. Particularly, the sheath may surround the distal end of the stent and a region of the stent adjacent the distal end. In this manner, the agent introduced into the stent is substantially prevented from leaking from the distal end. Alternatively, the sheath surrounds a greater portion of the stent in addition to the distal end. In one embodiment, the sheath extends around the stent elongate body from an open first end which is attached to a region of the stent adjacent the proximal end to a closed second end which surrounds the distal end of the elongate body.

The sheath essentially contains the cement and prevents leakage into surrounding bone. In embodiments, wherein the stent is used to bridge a fracture in the bone, the stent may be introduced through a cavity of the bone and across the fracture site. The stent may then receive the bone cement. The sheath substantially prevents the bone cement from leaking out of the stent and through the fracture site. It will be appreciated that the sheath may be formed around various parts of the stent depending upon the location of fracture site. For example, the sheath may only cover 10% of the length of the elongate body. Alternatively, the sheath may surround 20%, 30%, 40%, 50%, 60%, 70%, 80% 90% or the entire length of the elongate body.

The sheath may be made from a biocompatible resorbable material. Alternatively, the sheath may be non-resorbable. The sheath may be made from the same or a different material to that of the stent. The sheath may also include a therapeutic or radiopaque agent. The sheath may be attached to the stent body at one or more attachment points. The sheath may be attached by any number of means including gluing, suturing or moulding during the manufacture process. The attachment of the sheath to the stent preferably allows expansion of the stent from its compressed configuration to an expanded configuration. As such, the attachment points may allow a degree of movement of the sheath relative to stent elongate body as the latter expands radially. This may be achieved by providing a suture on the sheath, the suture encircling a strut of the elongate body and wherein the suture is at least partially moveable along the strut.

### Delivery System

The carrier device of the delivery system disclosed herein may include the bone stent or the introducer rod as herein disclosed.

Generally, however, the bone stent and introducer rod are configured for intramedullary deployment whereas the carrier device may be delivered to an external bone site or indeed to an area of tissue for grafting a particular bone and subsequently moved to a bone site. The carrier device is preferably delivered percutaneously to a desired site.

The delivery system herein disclosed, may be used to deliver any number of agents to a subject. In a preferred embodiment, the carrier device may be configured and the carrier substance may carry a suitable agent such as to promote bone growth. Where the carrier device and the carrier substance are made from a resorbable material, it is envisaged that the bone growth stimulated may result in new bone formed in the shape of the carrier device. Accordingly, the carrier device may be provided in any number of shapes as dictated by its ultimately use including the size and shape of the bone required.

The carrier device has a reservoir to receive and hold a carrier substance. By the term “reservoir” it is intended that this may form any part of an interior of the carrier device and may have one or more openings. In the embodiment, wherein the carrier device is a tubular structure, the reservoir may comprise a lumen of the tube. In this embodiment, the reservoir essentially has two open ends. In such an embodiment, it is envisaged that the carrier substance is of sufficient viscosity such that it does not migrate through the open-ended reservoir. In another embodiment, one or both of the ends of a tubular structure may be substantially closed. In the latter embodiment, the ends may be substantially closed by a mesh like structure which still allows the elution of an agent in the carrier substance and infiltration of into the reservoir by blood vessel and other cells.

The carrier substance is typically a high viscosity liquid carrier material (HVLCM).

In one embodiment, a combination of the size or apertures in the carrier device and the viscosity of the HVLCM causes the HVLCM to be contained within the carrier device reservoir, that is, it does not leak from the carrier device through the apertures despite its liquid state for a period of time.

By the term period of time, it is envisaged that the HVLCM may be substantially contained in the carrier device for a period of 1 week and 1 month. Alternatively, the HVLCM may be substantially contained in the carrier device up to 2 month, 3 months, 4 months, 5 months, 6 months, 7, months etc. by then having delivered the active member

The carrier device may be specifically configured to provide optimal surface tension of the HVLCM by way of, not only the material from which it is made, but also the arrangement and size of the apertures.

While aperture size has a bearing on the retention of the carrier substance in the reservoir, the apertures must also be of a size sufficient to allow infiltration of blood vessels and cells in the embodiments wherein the carrier device and the carrier substance are configured to promote bone growth.

The main body may comprise a stent-like structure as described above in relation to a bone stent. In addition to being a cylindrical structure, in this aspect the main body may comprise a substantially rectangular or square shape, such an embodiment being useful for intervertebral spinal fusions. In this embodiment, the stent may have a sufficient rigidity to resist compression by two adjacent vertebral bodies. Additionally, the main body may be substantially flattened to reduce the profile of the device for use in particularly small spaces. The main body may be custom made to conform to any desired shape.

The shape of the carrier device will likely depend upon its use. In one embodiment the carrier device is used to promote bone growth in a subject. The carrier device may be tailored to the shape of the bone to be repaired. For example, if the damaged or absent bone is in the jaw of a subject, the carrier device may comprise a substantially U-shaped main body. It is envisaged that a curved shape main body may have advantages in reconstruction of the jaw and other craniofacial surgeries, or to grow sections of bone in vivo for transfer to a recipient site in need of bone augmentation.

### Liquid Carrier Substance

The carrier substance may comprise a polymeric high viscosity liquid carrier material (HVLCM). One example of a suitable HVLCM for use with the bone stent or the delivery system described herein comprises sucrose acetate isobutyrate (SAIB). Alternatively, the HVLCM may comprise an esterified polysaccharide which is a homopolymer or copolymer comprising esterified sugar monomer units, where the sugar monomer units are esterified ketose or aldose sugars.

Alternatively, the sugar monomer units may be selected from the group comprising: esterified 2-deoxyribose, esterified 2-deoxy-D-ribose, esterified fructose, esterified galactose, esterified glucose, esterified 2-deoxy-glucose, esterified 2-deoxy-D-glucose, esterified arabinose, esterified lyxose, esterified, esterified ribose, esterified xylose, esterified ribulose, esterified xyulose, esterified allose, esterified altrose, esterified mannose, esterified gulose esterified, esterified idose, esterified talose, esterified psicose, esterified sorbose, esterified tagatose, or a mixture thereof may be used.

In one embodiment, the HVLCMs are phase-transitioning, wherein they form semi-solid depots under physiological conditions, i.e. in vivo.

The HVLCMs may be biodegradable. Further, the rate of release of an agent from an HVLCM carrier may be controlled by way of the choice of sugar monomer in the HVLCM, the type and quantity of ester groups on the HVLCM and the molecular weight of the HVLCM.

In one embodiment, the HVLCMs disclosed do not crystallise neat under ambient or physiological conditions.

The HVLCM disclosed may comprise an esterified polysaccharide with a viscosity of at least 5,000 mPa·s at 37° C. (and optionally at least 10,000, 15,000, 20,000, 25,000 or even about 50,000 mPa·s at 37° C.). The viscosity of the substance may constitute the property of the substance which partly determines the rate of elution of an agent carried therein to a surrounding bone.

In order to administer an HVLCM disclosed herein or allow the HVLCM to release an agent contained therein, for example a drug encapsulated therein, the HVLCM can be mixed with a solvent in order to reduce the viscosity.

The substance may further include pharmaceutically acceptable ingredients including, but not limited to: pharmaceutically acceptable carriers, diluents, excipients, adjuvants, fillers, buffers, preservatives, anti-oxidants, lubricants, stabilisers, solubilisers, surfactants (e.g., wetting agents), chelators, adjuvants, penetration enhancers or a mixture thereof.

### Agents

Any of the disclosed aspect and embodiments above may deliver an agent to a subject.

An agent may be present in an amount sufficient to deliver to a host subject an effective amount of the agent required to achieve a desired effect. The amount of agent, in particular bioactive agent, incorporated into the substance depends upon a desired release profile, the concentration of agent required for an effect such as a biological effect, and the desired period of release of the agent.

Concentrations of agent may be dependent on factors such as: absorption, inactivation, and excretion rates of the agent. Dosages may be adjusted by a clinician to take into consideration factors such as the severity of a particular condition.

The term “agent” refers to any substance to be delivered to a subject and includes bioactive agents. Bioactive agents as used herein refers to an organic molecule including a drug, peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including CDNA, or RNA, or a fragment thereof), nucleotide, nucleoside, oligonucleotides (including antisense oligonucleotides), gene, lipid, hormone, vitamin, including vitamin c and vitamin E; inorganic compound, or combination thereof, that causes a biological effect when administered in vivo to a subject, for example a human being or an animal, or on a subject, for example where the bioactive agent is delivered topically.

The term drug, as used herein, refers to any substance used internally or externally as a medicine for the treatment, cure, or prevention of a disease or disorder, and includes but is not limited to immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropie peptides, nonsteroidal and steroidal anti-inflammatory compounds, antipsychotics, and radiation absorbers, including UV-absorbers.

### Osteoinductive Agent

In one embodiment the composition or carrier disclosed herein, comprises a bioactive agent that is an osteoinductive agent. The osteoinductive agent may be selected from a group comprising: an osteogenic protein, or a growth factor, or a member or the TGF-beta superfamily, or a mixture thereof. The osteogenic protein may comprise bone morphogenetic protein (BMP), including human bone morphogenetic proteins (rhBMPs). Examples of BMPs include, but is not limited to: BMP-2, BMP-4, BMP-6, BMP-7 (OP-1), and BMP-9, rhBMP-1, rhBMP-2, rhBMP-3, rhBMP-4, rhBMP-5, rhBMP-6, rhBMP-7, rhBMP-8a, rhBMP-8b, rfiBMP-9, rhBMP-10, rhBMP-15, noggin resistant BMPs, or a combination thereof.

The osteoinductive agent may be a BMP approved for human use which is rhBMP-2, or rhBMP-7.

The osteoinductive agent may be rhBMP-2 which is present in an amount of about 0.1 to about 40 mg, preferably about 1 to about 12 mg. For example, the osteoinductive agent is rhBMP-2 and is present in a dose of about 0.1 to about 5 mg per mL; or is present in a dose of about 1 to about 2 mg per mL.

In one embodiment the osteoinductive agent is rhBMP-7 and is present in an amount of about 0.1 to about 40 mg, preferably about 1 to about 12 mg. For example, the osteoinductive agent is rhBMP-7 and is present in a dose of about 0.1 to about 5 mg per mL; or is present in a dose of about 1 to about 2 mg per mL.

In another embodiment the osteoinductive agent is a growth factor. Examples of growth factors include, but are not limited to: platelets/platelet derived growth factor (PDGF), vascular endothelial growth factor (VEGF), Myostatin (GDF-8), insulin-like growth factor (IGF), and/or a member of the TGF-beta superfamily such as TGF-beta1, TGF-beta2, TGF-beta3, growth and differentiation factors (GDFs), fibroblast growth factor-1 (FGF-1), fibroblast growth factor-2 (FGF-2), other fibroblasts growth factors, Growth/differentiation factor 5 (GDF-5) hepatocyte growth factor (HGF), epidermal growth factor (EGF) activins, inhibins, follistatin or other specific activators of these pathways. In another embodiment a mix of growth factors may be derived from human bone or other tissue, or from the products of cultured cells.

### Anti-Resorptive Agent

In one embodiment the agent may comprise an anti-resorptive agent. Preferably the anti-resorptive agent has synergy with any osteoconductive agents present in the composition. Examples of anti-resorptive agents include, but are not limited to: bisphosphonates including: zoledronic acid, pamidronic acid, ibandronic acid, etidronic acid, alendronic acid, risedronic acid, and tilurondic acid; IKK inhibitors; Osteoprotegerin (OPG); inhibitors of Cathepsin K, chloride ion channel blockers; antibodies directed against RANKL, SOST, and DKK1; and proton pump inhibitors, or a mixture thereof.

The anti-resorptive agent may, in a preferred embodiment, be selected from: zoledronic acid, OPG, and inhibitors of both IKK and Cathepsin K, anti-RANKL Ab, anti-SOST Ab, and anti DKK1 Ab.

In the embodiment wherein the anti-resorptive agent is Zoledronic acid it may is present in an amount of about 10 to about 1000 μg, preferably about 20 to about 500 μg. For other bisphosphonates, typical amounts would be in the order of about 1 to about 50 mg for Pamidronate, about 100 μg to about 1 mg for Aledronate, and about 40 μg to about 1 mg for Ibandronate, and about 50 μg to about 1 mg for Risedronate.

### Osteoconductive Agent

In one embodiment the agent may comprise an osteoconductive agent. The osteoconductive agent may comprise a ceramic particle. Examples of ceramic particles include, but are not limited to: ceramic particles derived from calcium phosphate including: hydroxyapatite, tricalcium phosphate (α and β), tetracalcium phosphate, anhydrous dicalcium phosphate, monocalcium phosphate monohydrate, dicalcium phosphate dehydrate, heptacalcium phosphate, octocalcium phosphate, calcium pyrophosphate, oxyapatite, calcium metaphosphate, dahlite, carbonatoapatite, monocalcium phosphate anhydrous, amorphous calcium phosphate, calcium deficient hydroxyapatite, fluorapatite; a calcium silicate ceramic that includes: calcium orthosilicate, wollastonite, dicalcium silicate, diopside, and bioglass (any composition); or a calcium salt such as calcium sulfate (α-calcium sulfate hemihydrates, β-calcium sulfate hemihydrates, calcium sulfate dehydrate and mixtures thereof), plaster of paris; or a mixture thereof. A portion of the calcium content in the ceramic may be replaced by another divalent cation such as magnesium or strontium.

### Angiogenic Agent

In another embodiment the agent is an angiogenic agent. Examples of angiogenic compounds include, but are not limited to: VEGF, angiopoietin, erythropoietin (EPO), nicotinic acid, desferoxamine (DFO) and 2-deocyribose.

### Additional Bioactive Agents

In one embodiment, the compositions disclosed herein, preferably pharmaceutically acceptable compositions, comprise a bioactive agent that is known to be associated with bone and affect the delivery and presentation of growth factors to cells included or added. Examples include, but are not limited to: heparin and other glycosaminoglycans and their components, as well as specific binding proteins such as TGF-β binding protein.

In another embodiment, the agent may comprise cells. Examples of cells include, but are not limited to: bone forming cells such as progenitor cells, stem cells, derived from marrow, adipose or another tissue, and/or osteoblasts. The cells may also be derived from the blood of the patient, either monocytes or platelets. The cells may be from a subject who requires bone growth in their body; alternatively the cells may be from a cell line or from a suitable donor.

In yet another embodiment, the agent may comprise antibodies, including neutralizing antibodies, antibody Fcs, or antibody-based therapeutics that affect bone formation, resorption or repair. These include anti-sclerostin and anti-RANKL treatments that can promote bone formation and/or inhibit resorption.

In yet another embodiment, the agent may comprise a growth factor that promotes wound healing, mitogenesis or angiogenesis. These include but are not limited to IGFs (e.g. IGF-1), PDGF or platelets, APC (activated protein C) or related factors.

In yet another embodiment, the agent may comprise an antibiotic. Examples include, but are not limited to, cationic steroid antibiotics, cyclic lipopeptides, glycylcyclines, oxazolidinones and lipiarmycins. Other antimicrobials include ceragenins.

### Applications

The intramedullary bone device, stent or introducer rod disclosed herein may be used in a number of applications including stabilising a fractured bone. Bone fractures include long bone fractures such as to the femur, tibia, fibula, humerus, radius and ulna; and other fractures such as to the metacarpal bones of the hand and metatarsal bones of the feet, the clavicle.

In one embodiment the carrier device and carrier substance disclosed herein can be used for bone repair and bone tissue engineering. For example the stent and HVLCMs can be used for: fracture healing (particularly with open fractures), spinal fusion, correction of bone defects, promoting the osteointegration of bone implants including plates, screws, frames, and joint replacement implants, restoration of osteonecrotic bone (including Perthes disease of the hip), and restoration of osteoporotic bone. These could include closed fractures, open fractures, small bone defects, critical sized bone defects, stress fractures, scoliosis/spine fusion, osteonecrosis (including osteonecrosis of the hip). In addition, osteochondral defects and defects of the cartilage could also be treated, including injuries to joints and joint replacement.

In an embodiment of the invention when use in spinal fusions, the carrier device and carrier substance may be delivered percutaneously and anteriorly to a disc site. The disc may have been partially or completely removed prior to said delivery to the site. Alternatively the carrier device and the carrier substance may be delivered in a posterior and/or posterior-lateral compartment of the vertebra. The carrier device and carrier substance may be placed between a muscle layer and the posterior or posterio-lateral elements of the vertebra.

### BRIEF DESCRIPTION OF THE DRAWINGS

FIG. 1A is a side view of a stent and sheath of the disclosure;

FIG. 1B is a side view of a stent and sheath which has been filled with a cement;

FIG. 2A is a schematic view of a stent of the disclosure;

FIG. 2B is a schematic view of the stent of FIG. 2A, filled with cement;

FIG. 3 is a schematic view of the stent of FIG. 2A when implanted in a clavicle bone of a subject;

FIGS. 4A to 4D show side views of embodiments of an introducer rod of the present disclosure;

FIG. 5 shows a side view of an introducer gun of the disclosure with an exemplary introducer rod attached;

FIG. 6 is a side view of an intramedullary bone device of the present disclosure;

FIG. 7 is a sectional view of part of the intramedullary bone device of FIG. 6, including a view of an introducer rod;

FIG. 8 is a sectional view of a further part of the intramedullary bone device of FIG. 6, including a view of an introducer rod;

FIG. 9 is a sectional view of a first perforation region of the intramedullary bone device of the present disclosure;

FIG. 10 is an enlarged view of the region marked B shown in FIG. 9;

FIG. 11 is an enlarged view of the region marked A in FIG. 9;

FIG. 12A is an image of a femur with the intramedullary bone device of the disclosure inserted through the intramedullary canal;

FIG. 12B is a radiographic image of the femur of FIG. 12A;

FIGS. 13A to 13D are schematic depictions of a femur and the process of inserting an intramedullary bone device across a fracture of the femur;

FIG. 14A is a photograph image of an anterior view and posterior view of the fractured sawbone of Experiment 1;

FIG. 14B shows radiographs of the sawbone shown in the photograph of FIG. 14A in the anterior and posterior views; and

FIGS. 15A to 15C are graphs providing the results of Experiment 2.

### DETAILED DESCRIPTION OF EXEMPLARY EMBODIMENTS OF THE DISCLOSURE

In one embodiment, a bone stent 10 is made up of a series of struts 11 which define apertures 12.

The stent 10 is radially expandable from a compressed to an expanded configuration when deployed in a bone.

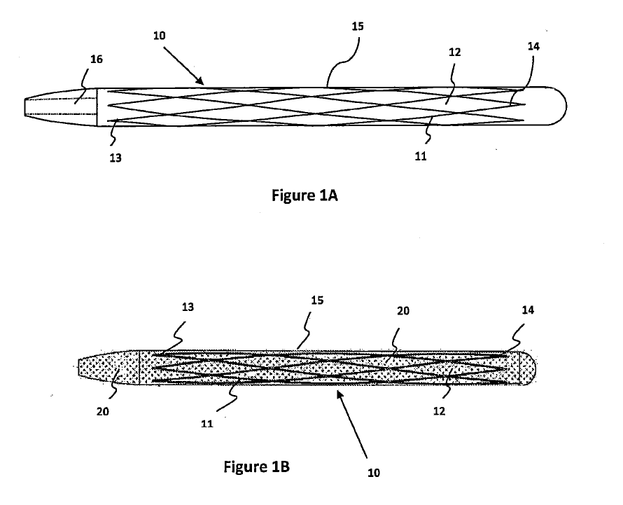

Stent 10 extends from a proximal end 13 to a distal end 14. In the embodiment shown in FIGS. 1A and 1B, for example, stent 10 also includes a sheath 15 which extends around the stent and covers the distal end 14. Proximal end 13 is not fully covered which allows for the delivery of a substance to the stent 10. This can be seen in FIG. 1A and FIG. 1B wherein sheath 15 has an inlet 16 at a region adjacent proximal end 13.

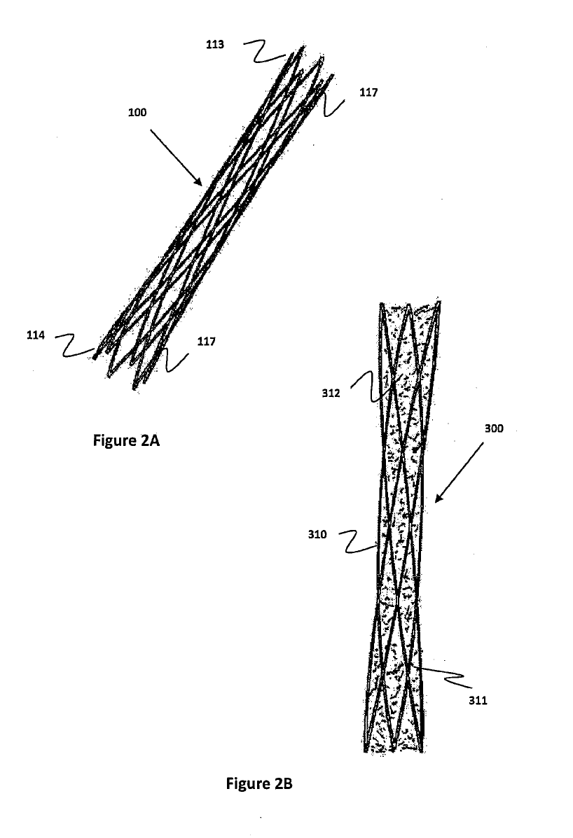

In one embodiment, stent 100 depicted in FIG. 2A has flared regions 117 at both the proximal end 113 and the distal end 114.

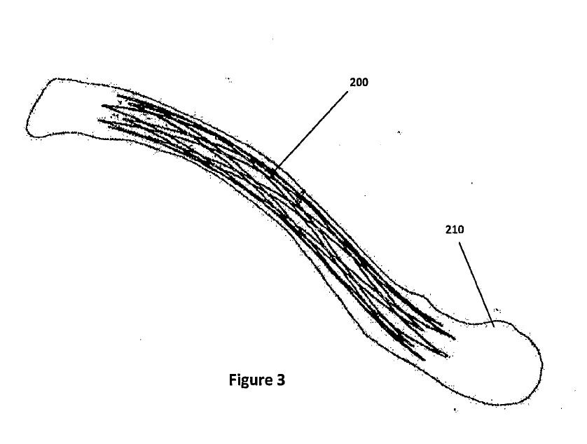

Stents 10, 100 are substantially straight whereas an example of a curved stent 200 deployed in a clavicle 210 is shown in FIG. 3.

As shown in FIG. 1B, the stent 10 and sheath 15 arrangement may be filled with a cement 20. The cement 20 is typically introduced through inlet 16 of sheath 15 and into the interior of the stent 10. The cement 20 is in a flowable form as it is introduced and thus migrates through apertures 12 to fill the stent 10 and the space around the stent 10 contained by sheath 15.

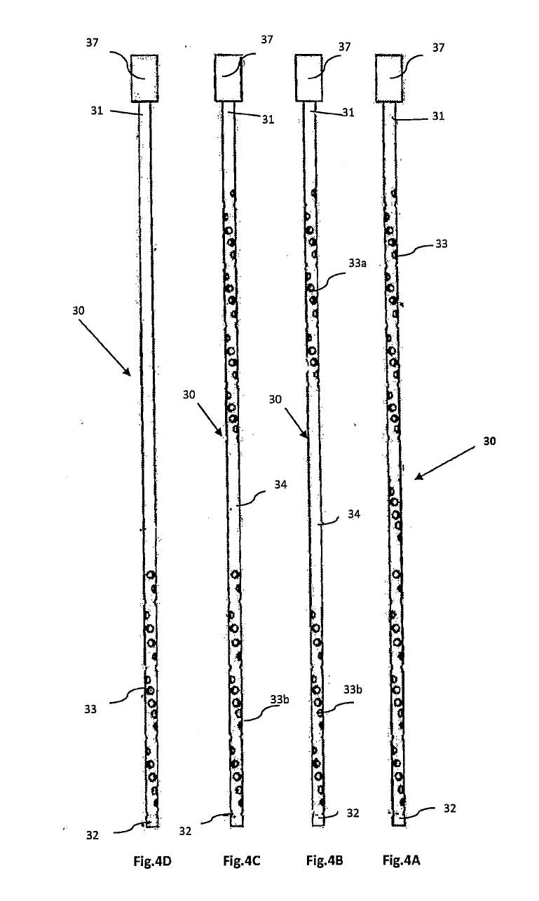

FIGS. 4A to 4D show a further embodiment of an introducer rod 30 for implantation in a bone of a subject. Introducer rod 30 comprises a tubular structure defining a lumen (not depicted) and which extends from a proximal end 31 to the distal end 32. The introducer rod 30 has a series of apertures 33 to allow fluid inserted into the lumen to exit the rod 30.

The apertures may be arranged a number of different way. For example, in FIG. 4A, the apertures are arranged helically along a majority of the length of rod 30. In FIG. 4B, an alternative embodiment is provided in which there are two separate groupings of apertures, 33a and 33b and a portion 34 of rod 10 which is devoid of apertures. In this embodiment, it may be advantageous to deploy the rod such that portion 34 is positioned at the region of fracture of a bone so that any fluid introduced into the rod 30 does not leak at this portion and through the fractured bone. The length of portion 34 may vary depending upon the size of bone and degree of fracture. As shown in FIG. 4C, the length of portion 34 is less than portion 34 of the device depicted in FIG. 4B.

In FIG. 4D only a length of rod 30 adjacent the distal end 32 comprises apertures 33.

The introducer rod may comprise an entry portal 37 to receive a fluid. In this embodiment, the entry portal comprises part of a luer lock system for attachment to a fluid source.

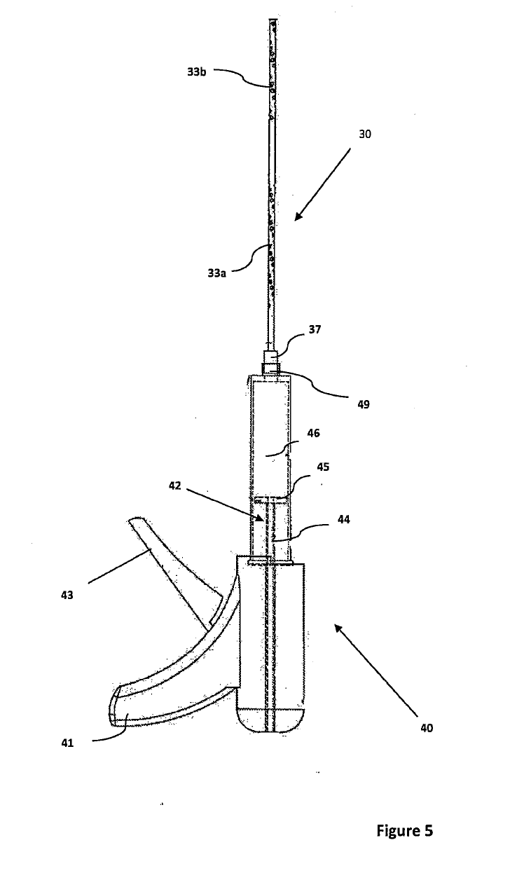

In FIG. 5, an introducer gun 40 is depicted which is shown coupled to a proximal end 31 of rod 30. The particular embodiment shown relates to the introducer rod 30 shown in FIG. 4C although any of the embodiments of 4A to 4D may be attached to such an introducer gun 40. The coupling is depicted as a luer lock connection with a luer component 49 of gun 40 connecting with the entry portal 37 but could be any form of coupling suitable for the purpose. The introducer gun 40 comprises a handle 41 and a plunger mechanism 42, the plunger mechanism 42 connected to an actuator lever 43 of handle 41. Plunger mechanism 42 comprises an elongate piston 44 and an end plunger head 45. The introducer gun also includes a barrel 46 which is dimensioned to hold a sufficient volume of fluid for introduction into the introducer rod 30.

When a user pulls actuator 43 in a direction of handle 41, the plunger head is driven distally towards the rod 30 to force a fluid in barrel 46 into the lumen of rod 30.

In embodiments, wherein the fluid is a bone cement, the coupling between rod 30 and actuator gun 40, may include a motor (not depicted) to cause rotation of the rod 30 around a longitudinal axis as the cement is introduced from the barrel and into the lumen of rod 30.

In another embodiment of the present disclosure, a carrier device 300 may be used to deliver a carrier substance to a desired location. In this embodiment the location may not be within a bone but could be a surface bone region or other area of the body. The carrier device may be any suitable configuration and could include, for example, the stent 310 of FIG. 2B. The carrier 300 is configured to receive and hold a carrier substance 311 which has a pre-determined viscosity to allow it to be retained in stent 310 for a preferred period of time.

The carrier substance 311 of this embodiment may be a high viscosity liquid carrier material (HVLCM).

In addition to the viscosity of the HVLCM, apertures 312 of stent 310 are sized and/or shaped to provide an optimal surface tension to retain the carrier substance 311 within stent 310.

In embodiments to promote bone growth or graft new bone, the HVLCM will be loaded with an agent or a plurality of agents which gradually elute to enhance bone growth.

### Intramedullary Bone Device

In an exemplary embodiment, the disclosure relates to an intramedullary bone device 400 which extends from a first end 401 to a second end 402 and has a sidewall 403 defining an internal lumen 404.

The sidewall 403 of the intramedullary device 400 comprises a first perforated region 405 adjacent the first end 401 and a second perforated region 406 adjacent the second end 402.

The first perforated region 405 comprises a plurality of first openings 407 in the sidewall 403 and the second perforated region 406 comprises a plurality of second openings 408 in the sidewall 403. The first 407 and second 408 openings are in fluid connection with the internal lumen 404.

The intramedullary bone device 400 further includes a first flow directing feature 420 associated with the first openings 407 and a second flow directing feature 440 associated with the second openings 408.

The first flow directing feature 420 directs the flow of a fluid from the internal lumen 404 through the first openings 407 in a first direction as shown by arrows 5a and 5b and the second flow directing feature 440 directs the flow of a fluid from the internal lumen 404 through the second openings 408 in a second direction which is different to the first direction, as shown by arrows 6a, 6b and 6c.

The intramedullary bone device 400 is tubular and extends along a first axis 7. The first perforated region 405 and the second perforated region 406 are separated by an intermediate region 409. Intermediate region 409 does not have openings in the sidewall 403.

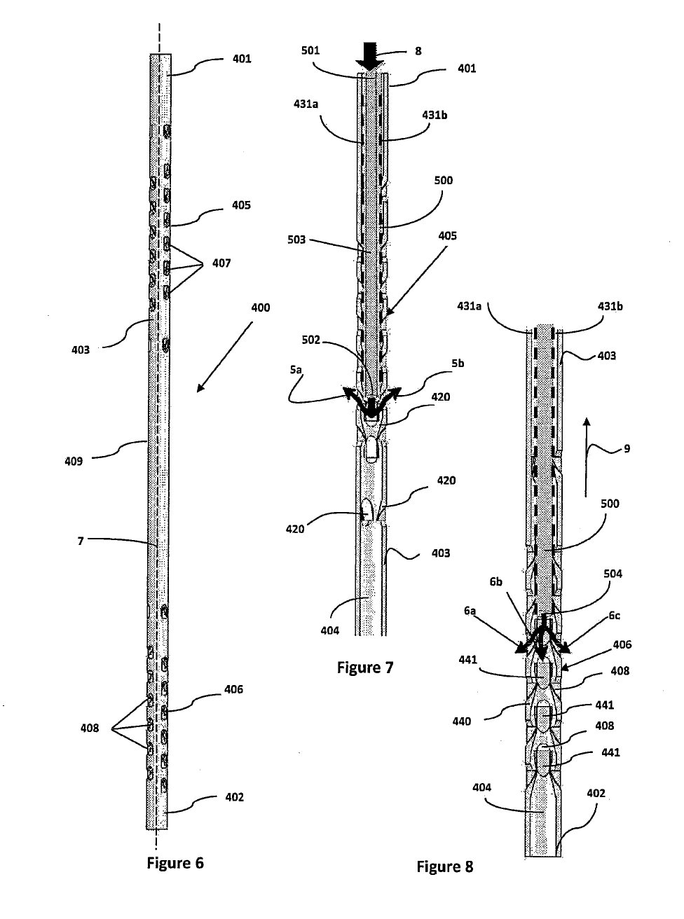

In the embodiment shown in FIG. 6, the first openings 407 and the second openings 408 are helically arranged along a length of sidewall 403 of the intramedullary bone device 400.

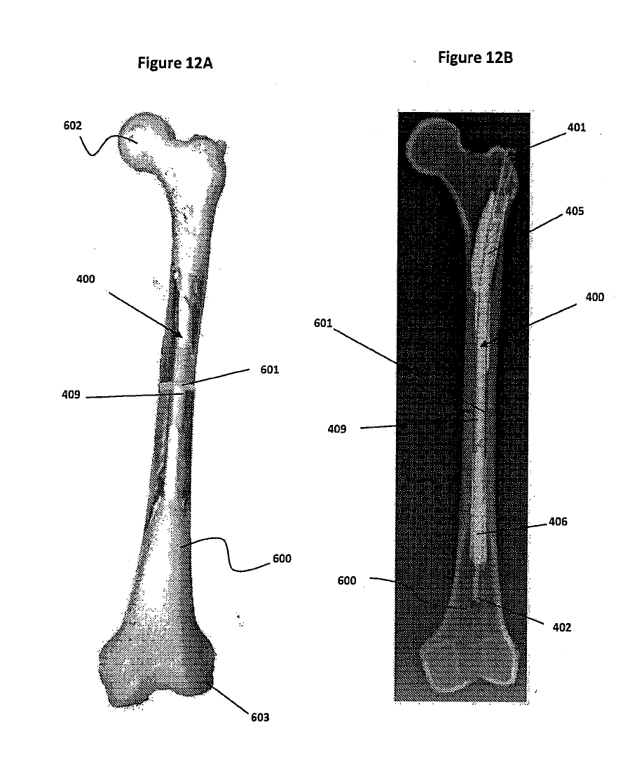

A first flow directing feature 420 as shown in FIGS. 10 and 11, for example, comprises part of the sidewall 403 of the intramedullary bone device 400 near an associated first opening 407.

Similarly, a second flow directing feature 440 as shown in FIG. 8 comprises part of the sidewall 403 of the intramedullary bone device 400 near an associated second opening 408.

The first flow directing feature 420 may comprise a first fluid guide member 421. First fluid guide member 421 extends inwardly and at an angle from an attachment with an internal surface of sidewall 403 and into internal lumen 404.

In one embodiment, first fluid guide member 421 depends from a connection end 422 connected to the sidewall. The connection end 422 in this embodiment comprises part of an upper edge 423 of an associated first opening 407.

The first fluid guide member 421 extends from the connection end 422 towards the second end 402 of the intramedullary bone device 400 and to a second end 424.

A first fluid guide member 421 as depicted in, for example FIG. 10, is substantially curved across its width and along its length from the connection end 422 to second end 424. This provides a curved fluid engagement surface 425. The curved fluid engagement surface 425 provides an increased surface area for subsequent bonding with a fluid.

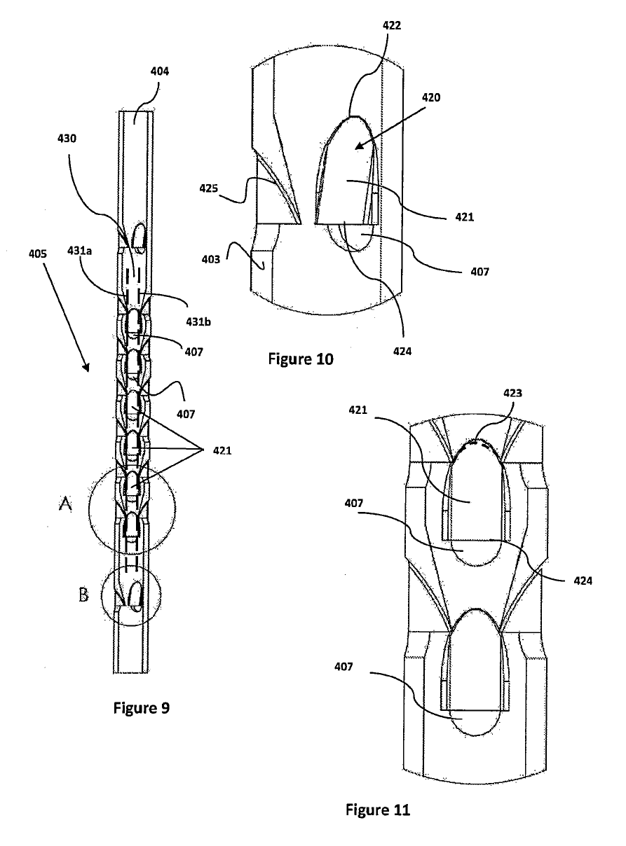

A plurality of first fluid guide members 421 may be arranged internally in the first perforated region 405 of the intramedullary bone device 400 as shown in, for example, FIG. 9. Each fluid guide member 421 is associated with a first opening 407.

The first fluid guide members 421 as arranged internally in FIGS. 7 and 9 together define a narrower passage 430 within the internal lumen 404 of the first perforated region 405. The narrow passage is shown as the passage between dashed lines 431a and 431b in FIGS. 7, 8 and 9.

The intramedullary bone device 400 may receive an introducer rod 500. The introducer rod 500 may be configured to connect to a fluid source and sized such that it is insertable into the intramedullary bone device 400 and into the internal lumen 404 at first end 401. As the introducer rod 500 is progressed toward second end 402 of intramedullary device 400, it is confined to the narrower passage 430 defined by the multiple first fluid guide members 421. In the embodiments shown in for example FIGS. 7 and 9, the narrower passage 430 is relatively central and thus centralizes the introducer rod 500 within the intramedullary bone device 400.

The first fluid guide members 421 as shown in the drawings are substantially identical in shape and/or orientation relative to each other to provide a uniform flow pattern of fluid therethrough. However, it is noted that the first fluid guide members 421 may comprise different shapes relative to each other and/or may extend at different angles relative to the sidewall 403.

The second flow directing feature 440, in one embodiment comprises a second fluid guide member 441. Second fluid guide member 441 is typically the same shape as the first fluid guide member 421. However, as noted from FIG. 8, the second fluid guide members 441 have a different orientation relative to the first fluid guide members 420 which are shown in FIG. 7.

In the depicted embodiments, the intramedullary bone device 400 is insertable in a medullary cavity of a fractured bone to stabilise the bone during healing of the fracture. To further stabilise the bone, the fluid of the disclosure is a bone cement which is introduced via introducer rod 500. The bone cement is introduced in a relatively flowable state but wherein the cement substantially hardens upon delivery.

The intramedullary bone device 400 is shown in FIGS. 12A and 12B stabilising a fracture of a femur 600. The femur 600 of this embodiment comprises a saw bone deliberately fractured at site 601 as part of an experiment to observe the flow of a cement through the device 400.

The intramedullary bone device depicted is a bioresorbable device and the cement used in FIGS. 12A and 12B is a bioabsorbable bone cement.

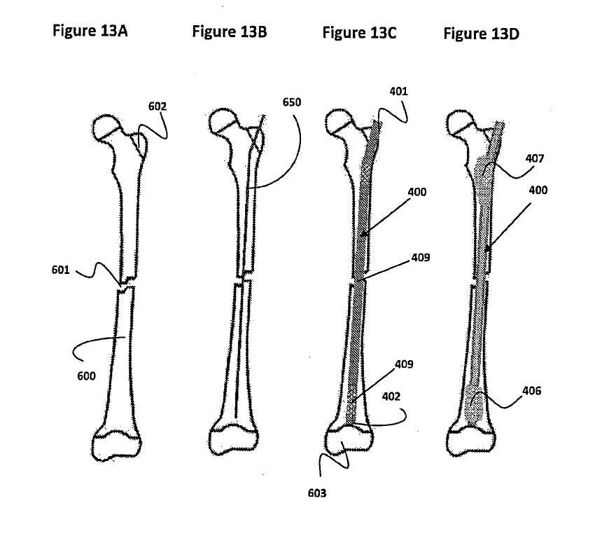

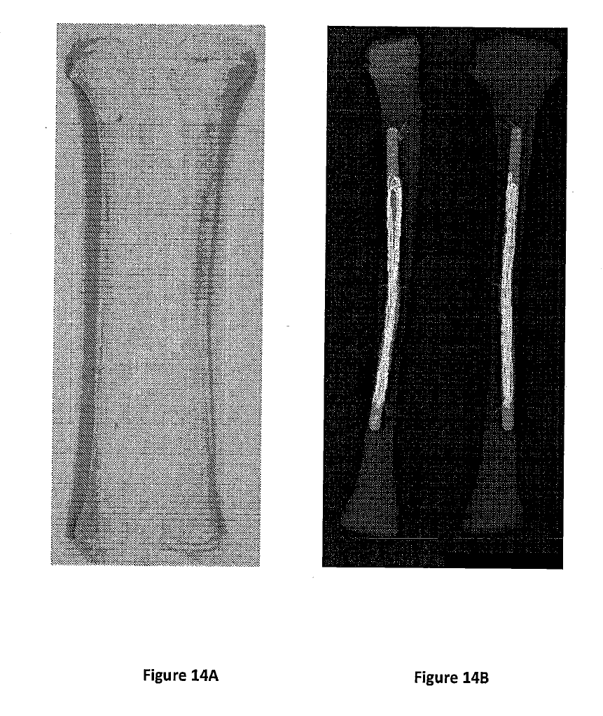

Intramedullary bone device 400 is implanted as shown in FIGS. 12A and 12B by drilling an entry hole in the greater trochanter 602 of the femur 600 and inserting a guidewire 650 through the intramedullary canal. The process is also schematically represented in FIGS. 13A to 13D. A reaming device may be inserted over the guidewire 650 (this step is not shown) to ream a passage through the intramedullary canal. The intramedullary device 400 is then inserted over the guidewire and advanced towards the distal epiphysis 603 of the femur 600. The second end 402 is positioned distally relative to the fracture site 601 while the first end 401 extends from the entry hole in the greater trochanter 602. Intermediate portion 409 spans the fracture site 601.

In one embodiment, prior to insertion of the intramedullary device 400 over the guidewire, an introducer rod 500 is inserted into the internal lumen 404 at the first end and advanced towards the second end 402 of the intramedullary bone device 400. Introducer rod 500 extends from a first end 501 to a second end 502. An inner lumen 503 is formed through introducer rod 500 from the first end 501 to the second end 502. First end 501 is connectable to a fluid source which, in the embodiment depicted in FIG. 7, comprises a source of cement shown as arrow 8.

Second end 502 comprises an exit portal 504 as shown, for example in FIG. 8. The cement introduced into the first end 501, flows through lumen 503 and eventually exits via exit portal 504.

The introducer rod 500 is typically inserted into the intramedullary bone device 400 until the exit portal 504 is positioned in the second perforated region 406. The exit portal 504 may be aligned with the furthest second openings 408, that is the second openings closest to the second end 402 of device 400.

Once the device 400 and introducer rod 500 are optimally positioned, a cement source is coupled to the first end 501 of introducer rod 500 and cement introduced such that it flows towards second end 502. As the cement starts to flow from exit portal 504 it is channeled by second fluid guide members 441 towards second openings 408. The positioning of second guide members 441 is such that the cement flows in the direction of arrows 6a, 6b and 6c. The direction of flow in the second perforated region is therefore towards the second end 402 of the intramedullary device 400. When is use, this is important because it directs the cement away from a fracture site such as 601 in FIG. 12B.

It can be seen from the radiographic image of FIG. 12B that the cement (showing as brighter white than the device 400 and femur 600) is localised around the second perforated region 406.

The introducer rod 500 is gradually retracted from the second end 402 of the intramedullary bone device 400 towards the first end 401. FIG. 8 shows the introducer rod 500 as it is being retracted towards the first end 401 of the intramedullary bone device 400 as indicated by arrow 9.

The intermediate region 409 of the intramedullary bone device 400 is positioned bridging fracture site 601 in FIG. 12B. The absence of openings in intermediate region 409 prevents the flow of cement to the fracture site 601 and instead the cement is contained within the internal lumen 404.

The introducer rod 500 is further retracted until exit portal 504 is positioned within the first perforated region 405. As the cement continues to flow from the exit portal 504, it is channeled by the first fluid guide members 421 in a direction shown by arrows 5a and 5b, that is, towards the first end 401 of the intramedullary bone device 400. This directing of the cement towards the first end 401 of the device 400 urges the cement away from the fracture site. Again, as can be seen from the X-ray in FIG. 12B, the cement introduced in the first perforated region 405 is substantially localised to said region and does not extend into fracture site 601.

The introducer rod 500 may then be removed fully from the intramedullary bone device 400 and the fracture held until the cement has substantially set.

### EXPERIMENTS

### Experiment 1

A pilot bench experiment was performed with a paediatric tibia sawbone (Pacific Research Laboratories, Inc. Washington USA). The sawbone was subjected to a bending force and fractured. A 6.5 mm medullary canal was created by drilling through a region of the sawbone adjacent a proximal region and an entry portal of 2 mm created proximally in the medial aspect of the metaphysis. A vent hole in the bone was created distally.

A metallic stent was inserted and a calcium phosphate cement injected through the entry portal, filling the medullary canal. After a 10 minute curing time the bone was seen to be well fixed.

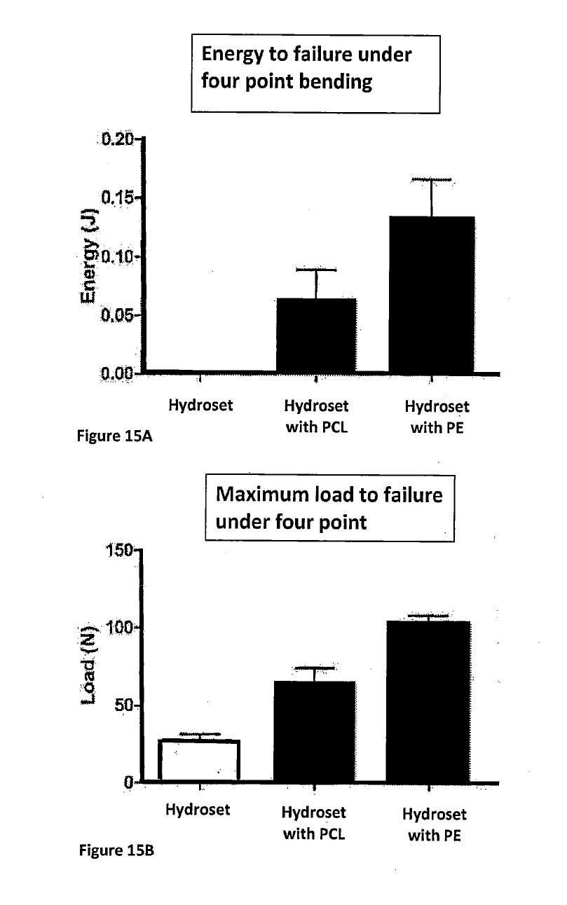

Radiographs were taken in the anterio-posterior and lateral projection and are shown in FIG. 14B. These clearly show the medullary canal filled with calcium phosphate cement, with the more dense loops of the metallic stent are seen crossing the fracture and reinforcing the cement. The pilot hole and venting hole can also be seen.

### Testing

Four point non-destructive bend testing was carried out at a rate of 2 mm/min to a limit of 60N with a 1 kN load cell on an Instron 5944 mechanical testing machine (Instron; Melbourne, Australia) and the stiffness of the fractured, treated sawbone with the cement filled stent in situ was 85% of the stiffness of an intact (unfractured) sawbone.

### Experiment 2

This experiment aimed to test the strength and stiffness of calcium phosphate cement against a polymer device filled with the cement.

Cylinders of calcium phosphate cement (Hydroset, Stryker) were produced and allowed to cure for 24 hours.

3 Groups were tested:

1. Hydroset alone

2. Hydroset cylinder surrounded by a polycaprolactone (PCL) intramedullary bone device as herein disclosed

3. Hydroset together with polyester (PE) mesh peripheral reinforcing

### Testing

Four point bend destructive testing was carried out at a rate of 2 mm/min with a 1 kN load cell on an Instron 5944 mechanical testing machine (Instron; Melbourne, Australia). Inner span length was 10 mm with outer 30 mm.

### Energy to Failure Under Four Point Bending

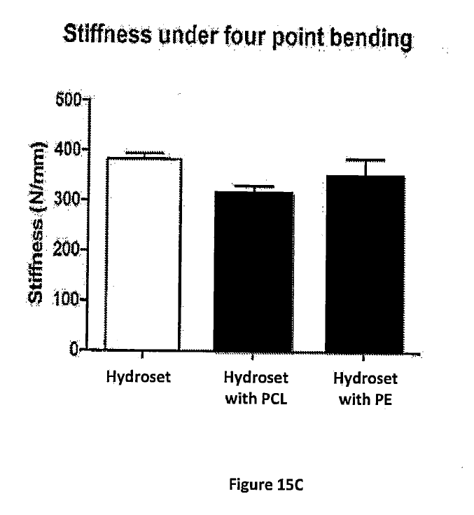

The brittle calcium phosphate cement of group 1 failed relatively very early, absorbing only a small amount of energy in bending before failure. As shown in FIG. 15A, the increase in energy to failure in the PCL introducer of group 2 in situ was 10 fold higher than group 1, and the group 3 combination of mesh and hydroset provided a further 2.5 fold increase in energy to failure.

### Maximum Load to Failure Under Four Point Bending

As shown in FIG. 15B, the maximum load to failure was increased over two-fold above the group 1 results and by a factor of four for group 3.

### Stiffness Under Four Point Bending

The stiffness results as shown in FIG. 15C demonstrate the brittle nature of the calcium phosphate cement of group 1. While stiff, its energy to failure is very low causing it to easily break.

### Post Testing

The calcium phosphate of group 1 behaved in a chalk like manner and was separated into pieces. Both examples of group 2 and group 3 kept their structure and were still able to take load.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An intramedullary bone device extending from a first end to a second end and having a sidewall defining an internal lumen, said sidewall comprising a first perforated region at or adjacent to the first end and a second perforated region at or adjacent the second end, said first perforated region comprising first openings in the sidewall and the second perforated region comprising second openings in the sidewall, the first and second openings in fluid communication with the internal lumen, wherein the device further includes a first flow directing feature associated with at least one of the first openings and a second flow directing feature associated with at least one of the second openings and wherein the first flow directing feature directs the flow of a fluid from the internal lumen through the first openings in a first direction and the second flow directing feature directs the flow of a fluid from the internal lumen through the second openings in a second direction which is different to the first direction.

2. The intramedullary bone device of claim 1 further comprising an intermediate region between the first and second perforated regions wherein the intermediate region is devoid of openings in the sidewall.

3. The intramedullary bone device of claim 1 wherein at least some of the first openings and/or second openings are helically arranged around the sidewall.

4. The intramedullary bone device of claim 1, wherein a first flow directing feature comprises part of the sidewall of the intramedullary bone device near an associated first opening.

5. The intramedullary bone device of claim 4 wherein the part of the sidewall extends into the internal lumen of the intramedullary bone device to form a first fluid guide member.

6. The intramedullary bone device of claim 5, wherein the first fluid guide member extends into the internal lumen at an angle relative to the sidewall of the intramedullary bone device.

7. The intramedullary bone device of claim 5 wherein the first fluid guide member extends from a connection end connected to the sidewall to a second free end.

8. The intramedullary bone device of claim 7, wherein the connection end is connected to part of the sidewall defining an associated first opening and wherein the first fluid guide member extends from said connection end towards the second end of the intramedullary bone device to define a flow channel in the internal lumen to the associated first opening.

9. The intramedullary bone device of claim 5, comprising a plurality of first fluid guide members arranged to define an internal passage within the internal lumen of the first perforated region.

10. The intramedullary bone device of claim 9, wherein the internal passage is substantially centrally positioned within the internal lumen and sized to receive a fluid introducer member.

11. The intramedullary bone device of claim 1 wherein at least one second flow directing feature comprises part of the sidewall of the intramedullary bone device near an associated second opening.

12. The intramedullary bone device of claim 11, wherein the second fluid guide member extends into the internal lumen at an angle relative to the sidewall of the intramedullary bone device.

13. The intramedullary bone device of claim 10 wherein the second fluid guide member extends from a connection end connected to the sidewall to a second free end.

14. The intramedullary bone device of claim 13, wherein the connection end is connected to part of the sidewall defining an associated second opening and wherein the second fluid guide member extends from said connection end towards the first end of the intramedullary bone device to define a flow channel in the internal lumen to the associated second opening.

15. The intramedullary bone device of claim 10, comprising a plurality of second fluid guide members arranged to define an internal passage within the internal lumen of the second perforated region.

16. The intramedullary bone device of claim 15, wherein the internal passage is substantially centrally positioned within the internal lumen and sized to receive a fluid introducer member.

17. The intramedullary bone device of claim 1, when made from a polymeric material.

18. The intramedullary bone device of claim 17, wherein the polymeric material is a biocompatible resorbable polymer.

19. The intramedullary bone device of claim 18, wherein the biocompatible, resorbable polymer comprises one or more of poly(α-hydroxy esters), poly(caprolactone), poly(p-dioxanone), poly(trimethylene carbonate), poly(oxaesters), poly(oxaamides), and their co-polymers and blends.

20. The intramedullary bone device of claim 18, wherein the biocompatible resorbable polymer comprises one or more of tyrosine derived poly amino acids, poly(arylates), poly(imino-carbonates), phosphorous containing polymers, poly(ethylene glycol) [PEG] based block co-polymers, poly(α-malic acid), poly(ester amides), or polyalkanoates.

21. The intramedullary device of claim 18, wherein the device is made from a co-polymer of poly(lactide), and poly(glycolide).

22. The intramedullary bone device of claim 21 further including trimethyl carbonate.

23. The intramedullary bone device of claim 18, wherein the biocompatible resorbable polymeric material is blended with a pH modifying agent which modifies the pH profile of the intramedullary bone device as it is resorbed.

24. The intramedullary bone device claim 18, wherein the biocompatible resorbable polymeric material further comprises bioglass.

25. The intramedullary bone device of claim 1 wherein the fluid is a biocompatible, resorbable fluid.

26. The intramedullary bone device of claim 25, wherein the fluid comprises a biocompatible, resorbable bone cement.

27. The intramedullary bone device of claim 26, wherein the resorbable bone cement includes calcium.

28. The intramedullary bone device of claim 27, wherein the resorbable bone cement comprises calcium phosphate.

29. The intramedullary bone device of claim 27, wherein the resorbable bone cement comprises one or more of dicalcium phosphate, tricalcium (α and β) phosphate, or tetracalcium phosphate hydroxyapatite.

30. The intramedullary bone device of claim 27, wherein the resorbable bone cement comprises one or more of heptacalcium phosphate, octocalcium phosphate, calcium pyrophosphate, oxyapatite, calcium metaphosphate, dahlite, carbonatoapatite, monocalcium phosphate anhydrous, amorphous calcium phosphate, calcium deficient hydroxyapatite, fluorapatite; a calcium silicate ceramic, a calcium sulfate salt, or plaster of Paris.

31. The intramedullary bone device of claim 27, wherein the resorbable bone cement has a resorbing pH profile which is basic.

32. An intramedullary bone system comprising:
a device which extends from a first end to a second end and comprises a sidewall defining an internal lumen, said sidewall including a first perforated region adjacent said first end and a second perforated region adjacent the second end, said first perforated region comprising first openings in the sidewall and the second perforated region comprising second openings in the sidewall, the first and second openings in fluid communication with the internal lumen, wherein the device further includes a first flow directing feature associated with the one or more first openings and a second flow directing feature associated with the one or more second openings and wherein the first flow directing feature directs the flow of a fluid from the internal lumen through the first openings in a first direction and the second flow directing feature directs the flow of a fluid from the internal lumen through the second openings in a second direction which is different to the first direction;
a fluid introducer member positioned substantially within the internal lumen of the device, said fluid introducer member comprising a first end having an entry portal to receive the fluid, a second opposed end having at least one exit portal, and an internal channel fluidly connecting the entry and exit portals.

33. A method of stabilising and/or securing a bone including;
preparing an entry point in the bone;
inserting a guidewire into the intramedullary canal of the bone;
reaming a length of the intramedullary canal over the guidewire;
advancing a device over the guidewire, the device extending from a first end to a second end and having a sidewall defining an internal lumen, said sidewall comprising a first perforated region adjacent the first end and a second perforated region adjacent the second end, the first perforated region comprising first openings in the sidewall and the second perforated region comprising second openings in the sidewall, the first and second openings in fluid communication with the internal lumen wherein the device further includes a first flow directing feature associated with the one or more first openings and a second flow directing feature associated with the one or more second openings;
withdrawing the guidewire when the device is desirably positioned within the bone;
introducing a fluid into the internal lumen of the device such that the first flow directing feature directs the flow of the fluid from the internal lumen through the first openings in a first direction and the second flow directing feature directs the flow of the fluid from the internal lumen through the second openings in a second direction which is different to the first direction.

34. A method of stabilising and/or securing a bone including;
preparing an entry point in the bone;
inserting a guidewire into the intramedullary canal of the bone;
reaming a length of the intramedullary canal of the bone over the guidewire;
advancing an intramedullary bone system over the guidewire, the intramedullary bone system comprising;
an intramedullary device extending from a first end to a second end and having a sidewall defining an internal lumen, said sidewall comprising a first perforated region adjacent the first end and a second perforated region adjacent the second end, the first perforated region comprising first openings in the sidewall and the second perforated region comprising second openings in the sidewall, the first and second openings in fluid communication with the internal lumen wherein the device further includes a first flow directing feature associated with the one or more first openings and a second flow directing feature associated with the one or more second openings; and
a fluid introducer member positioned substantially within the internal lumen of the intramedullary bone device, said fluid introducer member comprising a first end having an entry portal to receive the fluid, a second end having at least one exit portal, and an internal channel fluidly connecting the entry and exit portals and wherein the exit portal of the fluid introducer member is positioned within the second perforated region of the intramedullary bone device;
withdrawing the guidewire when the intramedullary bone system is desirably positioned within the bone;
introducing a fluid into the internal channel of the fluid introducer member such that said fluid flows from the exit portal of the fluid introducer member and is directed by at least one second flow directing feature through at least one second opening in a second direction;
withdrawing the fluid introducer member until the exit portal is positioned within the first perforated region of the intramedullary bone device such that the fluid flows from the exit portal of the fluid introducer member and is directed by at least one first flow directing feature through at least one first opening in a first direction, the first direction being different to the second direction.

35. The method of claim 34, wherein the intramedullary bone device stabilises and/or secures a fracture in a bone of a subject.

36. The method of claim 34, wherein the bone includes the femur, tibia, fibula, humerus, radius or ulna.

37. The intramedullary bone system of claim 32, wherein the fluid is a biocompatible, resorbable fluid.

38. The intramedullary bone system of claim 37, wherein the fluid comprises a biocompatible, resorbable bone cement.

39. The intramedullary bone system of claim 38, wherein the resorbable bone cement includes calcium.

40. The intramedullary bone system of claim 38, wherein the resorbable bone cement comprises calcium phosphate.

41. The intramedullary bone system of claim 38, wherein the resorbable bone cement comprises one or more of dicalcium phosphate, tricalcium (α and β) phosphate, or tetracalcium phosphate hydroxyapatite.

42. The intramedullary bone system of claim 38, wherein the resorbable bone cement comprises one or more of heptacalcium phosphate, octocalcium phosphate, calcium pyrophosphate, oxyapatite, calcium metaphosphate, dahlite, carbonatoapatite, monocalcium phosphate anhydrous, amorphous calcium phosphate, calcium deficient hydroxyapatite, fluorapatite, a calcium silicate ceramic, a calcium sulfate salt, plaster of Paris, or a mixture thereof.

43. The intramedullary bone system of claim 38, wherein the resorbable bone cement has a resorbing pH profile which is basic.

44. The method of claim 34, further providing a suction force to assist in drawing the fluid from the fluid introducer member.

45. The intramedullary bone system of claim 42, wherein the resorbable bone cement comprises one or more of calcium orthosilicate, wollastonite, dicalcium silicate, diopside, bioglass (any composition), α-calcium sulfate hemihydrates, β-calcium sulfate hemihydrates, calcium sulfate dehydrate or a mixture thereof.

46. The intramedullary bone device of claim 30, wherein the resorbable bone cement comprises one or more of calcium orthosilicate, wollastonite, dicalcium silicate, diopside, bioglass (any composition), α-calcium sulfate hemihydrates, β-calcium sulfate hemihydrates, calcium sulfate dehydrate or a mixture thereof.

47. The intramedullary bone device of claim 20, wherein the biocompatible, resorbable polymer comprises one or more of poly(DTH carbonates), poly(phosphoesters), poly(phosphazenes), PEG-poly(lactic acid), PEG-poly(propylene glycol), PEG-poly(butylene terephthalate), poly(hydroxybutyrate (HB) copolymers, or poly(hydroxyvalerate) (HV) copolymers.

48. The intramedullary bone device of claim 19, wherein the biocompatible, resorbable polymer comprises poly(lactide) or poly(glycolide).
